(19) United States
(12) Patent Application Publication
(10) Pub. No.: US 2004/0101829 A1
(43) Pub. Date: May 27, 2004

(54) Protein that interacts with lipids and methods for treating hyperlipidemia

(75) Inventors: Jack T. Stapleton, Iowa City, IA (US); Sabina Wuenschmann, Troy, VA (US)

Correspondence Address: FULBRIGHT & JAWORSKI L.L.P., A REGISTERED LIMITED LIABILITY PARTNERSHIP, 600 CONGRESS AVENUE, SUITE 2400, AUSTIN, TX, 78701-3271, US

(73) Assignee: The University of Iowa Research Foundation

(21) Appl. No.: 10/445,724

(22) Filed: May 27, 2003

Related U.S. Application Data
(60) Provisional application No. 60/392,158, filed on Jun. 28, 2002.

(51) Int. Cl. C12Q 1/70

(52) U.S. Cl. 435/5

(57) ABSTRACT

The present invention provides methods of identifying inhibitors of HCV infection. The invention further describes methods of preventing and treating HCV infection in a subject. The invention also describes methods for reducing LDL levels in a subject.

The present application claims benefit of U.S. Provisional Serial No. 60/392,158, filed Jun. 28, 2002, the entire contents of which are hereby incorporated by reference.

The government owns rights in the present invention pursuant to grant number AA12671 from the National Institute of Health.

### BACKGROUND OF THE INVENTION

1. Field of the Invention

The present invention relates generally to the fields of cardiology and virology. More particularly, it concerns methods for using Hepatitis C Virus (HCV) E2 glycoprotein to reduce Low Density Lipoprotein (“LDL”) levels in a subject. In other embodiments, it concerns identifying inhibitors of HCV infection in a subject.

2. Description of Related Art

A. Heart Disease

Atherosclerosis is the leading cause of death in the United States with over 800,000 deaths per year (U.S. Pat. No. 5,902,831). Epidemiologic studies have shown that a large percentage of those afflicted have an elevation in blood low density lipoprotein (LDL) levels. LDL carries cholesterol from the liver to body tissues. An elevated cholesterol level (hypercholesterolemia) is commonly associated with an elevation in LDL levels. High blood cholesterol levels, specifically LDL-cholesterol, increase risk for coronary heart disease (CHD), whereas lowering total cholesterol and LDL-cholesterol levels reduces CHD risk.

Numerous pharmaceutical agents have been developed to treat or prevent atherosclerosis and its complications by controlling abnormally high blood LDL levels or lowering cholesterol levels. Often used pharmaceutical agents include nicotinic acid, clofibrate, dextrothyroxine sodium, neomycin, beta-sitosterol, probucol, cholestyramine and HMG-CoA reductase inhibitors, such as lovastatin and simvastatin. Unfortunately, many of these pharmaceutical agents often cause acute side effects in patients. Examples of these side effects may include intense cutaneous flush, pruritus, gastrointestinal irritation, hepatotoxicity, cardiac arrhythmias, nausea, weight gain, alopecia, impotence, abdominal pain, diarrhea, eosinophilia, skin rash, musculoskeletal pain, blurred vision, mild anemia, leukopenia, the enhancement of gallstones, constipation, and impaction (U.S. Pat. No. 5,902,831).

B. Hepatitis C Virus Infection

Hepatitis C virus (HCV) was discovered in 1989, and accounts for approximately 20% of acute hepatitis cases in the United States (Alter, 1997). About 80% of HCV infections become persistent, and 20% of these progress into chronic disease. Approximately 170 million people worldwide are infected with HCV (Conry-Cantilena et al., 1996). Due to the long period of time from infection until the development of serious liver disease, it is predicted that there will be a marked increase in liver disease resulting from HCV over the next 25 years (Williams, 1999; Seeff, 1997). In fact, surgery patients and others requiring blood transfusions, and especially those having suppressed immune systems, resulting, for example, from drugs administered in connection with organ transplantation, are at risk of developing HCV infection, which is the primary cause of transfusion-associated hepatitis in the world today. It has been estimated that posttransfusion hepatitis C may be responsible for up to 3,000 annual cases of chronic active hepatitis or cirrhosis of the liver in the U.S. alone (U.S. Pat. No. 5,633,388). Hemodialysis patients, as well as intravenous drug abusers are other groups which are at risk for acquiring HCV infection.

Various clinical studies have been conducted with the goal of identifying pharmaceutical agents capable of effectively treating HCV infection in patients afflicted with chronic hepatitis C. These studies have involved the use of dideoxynucleoside analogues and interferon-alpha, alone and in combination therapy with other anti-viral substances (U.S. Pat. No. 5,633,388). Such studies have shown, however, that substantial numbers of the participants do not respond to this therapy, and of those that do respond favorably, a large proportion were found to relapse after termination of treatment.

HCV primarily replicates in the hepatocyte (Major et al., 1997), but is also found in association with a variety of peripheral blood cells (PBC's) (Major et al., 1997; Schmidt et al., 1997). Although controversial, it appears that HCV replicates to some extent in PBCs, and inefficient in vitro cultivation can be achieved in T- and B-cell lines (Major et al., 1997; Bartenschlager et al., 2000).

The mechanisms by which HCV attaches and enters cells has not been clear. Two cellular surface receptors have been shown to interact with HCV or the HCV envelope glycoprotein E2 in vitro, leading to speculation that either may represent the HCV cellular receptor (Pileri et al., 1998; Monazahian et al., 1999; Agnello et al., 1999; Flint et al., 1999; Wuenschmann et al., 2000). It has been shown that recombinant HCV E2 binds to human CD81 (Pileri et al., 1998; Flint et al., 1999; Flint and Maidens et al., 1999; Hadlock et al., 2000; Owsianka et al., 2001; Flint and McKeating, 2000; Petracca et al., 2000; Patel et al., 2000). CD81 is a member of the tetraspanin superfamily of cell surface molecules, and is expressed on virtually all nucleated cells (Levy and Maecker, 1998). Initial studies suggested that E2 binding to CD81 may be responsible for the binding of HCV to target cells in vivo. However, although E2 has repeatedly been shown to bind CD81, only two studies presented evidence that HCV particles derived from human serum bind to this surface molecule (Pileri et al., 1998; Hadlock et al., 2000).

The inventors have showed that, although HCV E2 binds specifically to CD81 (Wuenschmann et al., 2000), the binding of HCV particles purified from plasma was not inhibited by soluble CD81, and the extent of virus binding correlated with the level of LDLr expression (Wuenschmann et al., 2000). Additional lines of evidence argue that CD81 is not the HCV receptor. HCV E2 has a higher affinity for marmoset CD81 than human CD81, yet marmosets are not susceptible to HCV. The affinity for HCV E2 to CD81 was found to be significantly lower than predicted for a true viral receptor (Petracca et al., 2000). Using an RT-PCR based detection method, plasma-derived HCV and HCV E2 bound to U937 subcloned cells that lack expression of CD81 (Hamaia and Allain, 2001). These data suggest that CD81 is not the primary cell receptor for HCV.

Nevertheless, HCV E2 does interact with CD81, and the E2 regions involved in CD81 binding are highly conserved (Pileri et al., 1998; Flint et al., 1999; Flint and Maidens et al., 1999; Hadlock et al., 2000; Owsianka et al., 2001; Flint and McKeating, 2000; Petracca et al., 2000; Patel et al., 2000)), suggesting a functional role for CD81-E2 interactions in HCV replication (Pileri et al., 1998; Flint et al., 1999; Flint and Maidens et al., 1999; Hadlock et al., 2000; Owsianka et al., 2001; Flint and McKeating, 2000). The extremely low density of HCV found in gradient centrifugation of infectious serum suggested an association with VLDL and LDL (Hijikata et al., 1993; Bradley et al., 1991; Prince et al., 1996). Infectious virus was found at the same densities as VLDL and LDL and coprecipitated with LDL (Monazahian et al., 1999; Bradley et al., 1991; Prince et al., 1996; Thomssen and Thiele, 1993; Xiang et al., 1998). Subsequent studies (Monazahian et al., 1999; Bradley et al., 1991; Prince et al. 1996; Xiang et al., 1998) demonstrated an interaction between HCV or HCV-LDL complexes with the low density lipoprotein receptor (LDLr) (Wuenschmann et al., 2000; Prince et al., 1996; Thomssen and Thiele, 1993; Xiang et al., 1998; Thomssen et al., 1992).

HCV present in the plasma of infected people has also been shown to interact with very-low-density (VLDL) and low-density lipoproteins (LDL). The liver synthesizes VLDL which consists of triaglycerols, cholesterol, phospholipids and the apoprotein apoB-100, VLDL's released into the blood, where it acquires additional lipoproteins C_{II} and apoE from high-density lipoproteins (HDL). VLDL is digested by Lipoprotein Lipase (LPL), an enzyme found attached to capillary endothelial cells, to form intermediate density lipoproteins (IDL) and LDL, and apoB-100 is the only remaining apoprotein in LDL. The low-density lipoprotein receptor (LDLr) recognizes both apoE and apoB-100 and can therefore bind VLDL, IDL and chylomicron remnants in addition to LDL. (Marks et al., 1996).

HCV-RNA containing material in serum, presumably virus particles, separate into very low density particles (<1.06 g/cm³) by gradient sedimentation, suggesting that HCV associates with VLDL and LDL (Monazahian et al., 1999; Thomssen et al., 1993; Xiang et al., 1998; Prince et al., 1996; Bradley et al., 1991). In addition, particles with densities of 1.11-1.18 g/cm³ have been described (Xiang et al., 1998; Prince et al., 1996; Bradley et al., 1991; Hijikata et al., 1993). Chimpanzee infectivity studies demonstrated that the very low density HCV particles were highly infectious, whereas the particles of higher density were not infectious (Bradley, 2000). (Monazahian et al., 1999; Xiang et al., 1998; Prince et al., 1996; Bradley et al., 1991). Thomssen et al. (1993) showed that HCV coprecipitated with LDL and demonstrated an interaction of HCV or HCV-LDL complexes with the LDLr (Wuenschmann et al., 2000; Thomssen et al., 1993; Xiang et al., 1998; Prince et al., 1996; Thomssen et al., 1992).

Monazahian et al. (1999) demonstrated that expression of recombinant human LDLr in murine cells lacking human CD81 confirmed binding of HCV to these cells (Monazahian et al., 1999) and Agnello et al. (1999) demonstrated that HCV bound to and entered fibroblasts containing LDLr, but not LDLr deficient fibroblasts, using an in situ hybridization method (Agnello et al., 1999). Using flow cytometry, the inventors confirmed that plasma-derived HCV bound to cells expressing LDLr, but not to cells lacking the LDLr (Wuenschmann et al., 2000). No interactions between viral envelope proteins (E1 or E2) and the LDL receptor have been reported (Wuenschmann et al., 2000). However, Monazahian et al. (1999) found that in vitro translated HCV E1 and E2 proteins, labeled with ³⁵S-methionine co-precipitated with VLDL, LDL and HDL (Monazahian et al., 2000).

C. HCV E2 Glycoprotein

HCV E2 is the outer protein of the viral envelope and may participate in the binding of viruses to the target cells. The protein starts at amino acid 394 of the HCV polyprotein, and extends to amino acid 747. It has a hypervariable region at the amino terminus of the protein, and the carboxy terminus includes a transmembrane domain.

Due to the deficiencies in the prior art, there remains a need for more effective treatments to lower LDL levels in a subject. There also remains a need for new and useful methods of reducing or preventing HCV infection in a subject. The presently claimed invention overcomes the deficiencies in the prior art by disclosing new and useful methods for reducing LDL levels in a subject. The present invention also discloses new and useful methods of identifying HCV inhibitors and methods of treating HCV infection.

### SUMMARY OF THE INVENTION

In accordance with the present invention, there is provided a method for reducing LDL levels in a subject comprising administering to the subject an HCV E2 glycoprotein. The E2 glycoprotein may be substantially purified away from other HCV components. In other aspects, the E2 glycoprotein may be comprised in a non-replicative viral particle. In other embodiments, the subject may have a history of familial hypercholesterolemia. The HCV E2 glycoprotein may be administered intravenously orally, nasally, parenterally, or intramuscularly. In yet another aspect, there is provided a method for reducing LDL levels in a subject comprising administering in combination with said E2 glycoprotein, another agent effective in lowering LDL levels in a subject. The agent may be nicotinic acid, clofibrate, dextrothyroxine sodium, neomycin, the “statin” class of drugs (for example, cerivastatin, fluvastatin, atorvastatin, lovastatin, pravastatin, and simvastatin), beta-sitosterol, probucol, cholestyramine or HMG-CoA reductase inhibitors.

Another aspect of the present invention provides a method of identifying an E2 peptide that is effective in lowering LDL levels in a subject comprising, providing a candidate E2 peptide, plasma lipoprotein, and a target cell expressing an LDL receptor under conditions effective to allow the formation of an E2 peptide/plasma lipoprotein/LDL receptor complex and assaying internalization of E2 peptide/plasma lipoprotein/LDL receptor complex. An increase in plasma lipoprotein into target cell, as compared to internalization of plasma lipoprotein into target cell in the absence of the E2 peptide, identifies the E2 peptide as effective in lowering LDL levels in a subject. In other aspects, the E2 peptide is a produced by chemical, physical or enzymatic cleavage of a purified E2 glycoprotein. In other embodiments, the E2 peptide is a C-terminal truncated E2 molecule or a recombinant peptide. In still other aspects of this invention, the E2 peptide is chemically synthesized. In other embodiments, the plasma lipoprotein may be a low density lipoprotein, a high density lipoprotein, a very low density lipoprotein, or a chylomicron. In further embodiments, the subject may be a human, a dog, a cat, a mouse, a deer, a rabbit, or a cow. In other aspects of the invention, the internalization of the E2 peptide/plasma lipoprotein/LDL receptor complex into target cell is determined by labeling the plasma lipoprotein. The label may be a radio label, isotopic label, fluorescent label, chemiluminescent label, enzymatic label or any other label that is well known in the art.

In another aspect of the present invention, there is provided a method of identifying an inhibitor of Hepatitis C Virus (HCV) infection comprising providing isolated E2 glycoprotein and plasma lipoprotein; admixing a candidate substance with the E2 glycoprotein and plasma lipoprotein; and determining the binding of the E2 glycoprotein to plasma lipoprotein, wherein a reduction in E2 glycoprotein binding to plasma lipoprotein, as compared to binding in the absence of the candidate substance, identifies the candidate substance as an inhibitor of HCV infection. In another aspect of the present invention, the candidate substance may be an anti-E2 antibody. The antibody may be a monoclonal or polyclonal antibody. The inhibitor of HCV infection may be a small molecule, a peptide, a protein, a polypeptide, or any other compound, substance, or agent. In yet another aspect of the present invention, the binding of the E2 glycoprotein to plasma lipoprotein may be determined by gel electrophoresis, gel filtration chromatography, fluorescence quenching assay, flow cytometry, elisa, solid phase immunoassay, or confocal microscopy.

In another aspect of the present invention, there is provided a method of identifying an inhibitor of Hepatitis C Virus (HCV) infection comprising providing isolated E2 glycoprotein and plasma lipoprotein under conditions effective to allow the formation of an E2 glycoprotein/plasma lipoprotein complex; providing a target cell expressing an LDL receptor; admixing said E2 glycoprotein/plasma lipoprotein complex and said target cell in the presence of a candidate substance; and determining the binding of the E2 glycoprotein/plasma lipoprotein complex to LDL receptor, wherein a reduction in E2 glycoprotein/plasma lipoprotein complex binding to LDL receptor, as compared to binding in the absence of the candidate substance, identifies the candidate substance as an inhibitor of HCV infection. The candidate substance may be an anti-LDL receptor antibody. The antibody may be a monoclonal or polyclonal antibody. In other embodiments, the candidate substance may be an anti-E2 glycoprotein/plasma lipoprotein antibody. The antibody may be a monoclonal or polyclonal antibody. The inhibitor of HCV infection may be a small molecule, a peptide, a protein, a polypeptide, or any other compound, substance, or agent. The binding of the E2 glycoprotein/plasma lipoprotein complex to LDL receptor may be determined by gel electrophoresis, gel filtration chromatography, fluorescence quenching assay, flow cytometry, elisa, solid phase immunoassay, or confocal microscopy.

In yet another aspect of the present invention, there is provided a method of identifying an inhibitor of Hepatitis C Virus (HCV) infection comprising, providing isolated E2 glycoprotein, plasma lipoprotein, and a target cell expressing an LDL receptor under conditions effective to allow the formation of an E2 glycoprotein/plasma lipoprotein/LDL receptor complex; contacting the LDL-expressing cell with a candidate substance; and determining internalization of E2 glycoprotein/plasma lipoprotein/LDL receptor complex into target cell, wherein a reduction in internalization of E2 glycoprotein/plasma lipoprotein/LDL receptor complex into target cell, as compared to internalization of E2 glycoprotein/plasma lipoprotein/LDL receptor complex into target cell in the absence of the candidate substance, identifies the candidate substance as an inhibitor of HCV infection. The candidate substance may be a polyclonal or monoclonal antibody. The inhibitor of HCV infection may be a small molecule, a peptide, a protein, a polypeptide, or any other compound, substance, or agent.

In still another aspect of the present invention, there is provided a method of removing plasma lipoproteins from a blood sample comprising, providing isolated E2 glycoprotein attached to a support; contacting the support with the blood sample under conditions effective to allow the binding of plasma lipoprotein present in the blood sample to E2 glycoprotein; and separating plasma lipoprotein from E2 glycoprotein. The support may be a non-reactive solid support. The non-reactive solid support may be a nitrocellulose membrane, a bead support, or a glass support.

In another aspect of the present invention, there is provided a method of screening for an inhibitor of Hepatitis C Virus (HCV) infection comprising, providing purified E2 glycoprotein and plasma lipoprotein, admixing an E2 antibody with E2 glycoprotein and plasma lipoprotein under conditions effective to allow the formation of an E2 glycoprotein/plasma lipoprotein complex; and determining the binding of E2 glycoprotein to plasma lipoprotein, wherein a reduction in E2 glycoprotein binding to plasma lipoprotein, as compared to binding in the absence of E2 antibody, identifies the E2 antibody as an inhibitor of HCV infection. The binding of the E2 glycoprotein to plasma lipoprotein may be determined by gel electrophoresis, gel filtration chromatography, fluorescence quenching assay, flow cytometry, elisa, solid phase immunoassay, or confocal microscopy.

In yet another aspect of the present invention, there is provided a method of inhibiting Hepatitis C Virus (HCV) infection in a subject comprising administering an effective amount of an agent that inhibits the formation of an E2 glycoprotein/plasma lipoprotein complex or an E2 glycoprotein/plasma lipoprotein/LDL receptor complex. The agent may be a small molecule, peptide, protein, polypeptide, antibody, substance, or compound. The agent may be a polyclonal or monoclonal antibody. In other aspects of the present invention, the agent may be administered orally, intravenously, parenterally, or intramuscularly. In other embodiments, the agent may be formulated in an aqueous formulation or a salt formulation. In other aspects, the agent may be formulated as an ingestible tablet, capsule, elixir, suspension, syrup, or a wafer. In still other aspects, there is provided a method of inhibiting HCV infection, further comprising administering in combination with the agent that inhibits the formation of an E2 glycoprotein/plasma lipoprotein complex or the E2 glycoprotein/plasma lipoprotein/LDL receptor complex, another agent effective in treating HCV infection in a subject. The other agent effective in treating HCV infection may alpha interferon, ribavirin, or Peginterferon Alfa-2b. Potential treatments include drugs that inhibit viral uncoating (e.g., amantidine), and inhibitors of HCV replication enzymes (e.g., helicase inhibitors, polymerase inhibitors, protease inhibitors, etc.).

In still other aspects of the present invention, there is provided a method of inhibiting Hepatitis C Virus (HCV) infection in a subject comprising administering an effective amount of an agent that inhibits the internalization of E2 glycoprotein/plasma lipoprotein/LDL receptor complex into a target cell. The agent may be a small molecule, a peptide, protein, polypeptide, antibody, compound, or substance. The antibody may be a polyclonal or monoclonal antibody. In still other aspects, the agent may be administered orally, intravenously, parenterally, or intramuscularly. In other embodiments, the agent may be formulated in an aqueous formulation or a salt formulation. In other aspects, the agent may be formulated as an ingestible tablet, capsule, elixir, suspension, syrup, or a wafer. In other aspects of the present invention, there is provided a method of inhibiting HCV infection, further comprising administering in combination with said agent that inhibits the internalization of E2 glycoprotein/plasma lipoprotein/LDL receptor complex into a target cell, another agent effective in treating HCV infection in a subject. The other agent effective in treating HCV infection may be α-interferon, ribavirin, or Peginterferon Alfa-2b. Potential treatments include drugs that inhibit viral uncoating (e.g., amantidine), and inhibitors of HCV replication enzymes (e.g., helicase inhibitors, polymerase inhibitors, protease inhibitors, etc.).

In yet another aspect of the present invention, there is provided an inhibitor of Hepatitis C Virus (HCV) infection that reduces or prevents the formation of an E2 glycoprotein/plasma lipoprotein complex, or an E2 glycoprotein/plasma lipoprotein/LDL receptor complex. The inhibitor may be a small molecule, peptide, protein, polypeptide, antibody compound or substance. The antibody may be a polyclonal or monoclonal antibody. In another aspect of the present invention, there is provided an inhibitor of Hepatitis C Virus (HCV) infection that reduces or prevents the internalization of an E2 glycoprotein/plasma lipoprotein/LDL receptor complex into a target cell. The inhibitor may be a small molecule, peptide, protein, polypeptide, antibody compound or substance. The antibody may be a polyclonal or monoclonal antibody.

The following definitions are provided:

The use of the word “a” or “an” when used in conjunction with the term “comprising” in the claims and/or the specification may mean “one,” but it is also consistent with the meaning of “one or more,” “at least one,” and “one or more than one.”

The term “drug” as used herein is defined as a medicament or medicine which is used for the therapeutic treatment of a medical condition or disease. The drug may be used in combination with another drug or type of therapy. In one embodiment, the drug is effective for the treatment of HCV infection. In another embodiment, the drug is effective for reducing LDL levels in a subject.

The term “to treat” as used herein is defined as the practice of applying a treatment for a medical condition or disease. The treatment need not provide a complete cure and is considered effective if at least one symptom is improved upon or eradicated. Furthermore, the treatment need not provide a permanent improvement of the disease state or medical condition, although this is preferable.

The term “plasma lipoprotein” as used herein includes chylomicrons, very low density lipoproteins (VLDL), high density lipoprotein (HDL), and low density lipoproteins (LDL).

The term “candidate substance” as used herein is defined as any compound, small molecule, peptide, protein, antibody or any other substance that may inhibit HCV infection.

The term “inhibitor” as used herein is defined as a reduction or complete inhibition of HCV infection in a subject.

The term “subject” as used herein is defined to include any dog, cat, mouse, rabbit, cow, deer, mammal, human, or any other animal.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A and FIG. 1B: Binding of low density HCV to MOLT 4 cells. MOLT-4 cells were incubated with sucrosegradient purified HCV. Cell-bound virus was detected with anti HCV polyclonal antibody (FIG. 1A) or E2-specific monoclonal antibody 108 (FIG. 1B).

FIG. 1C: Binding of plasma-derived human LDL to MOLT-4 cells. MOLT-4 cells were incubated with sucrosegradient purified HCV or Mock preparations. Cell-bound LDL was detected with a monoclonal anti-LDL antibody.

FIG. 2: Interaction of E2 with LDL. Increasing amounts of LDL were incubated with purified E2 protein at 4° C. and allowed to bind to mouse monoclonal anti-apolipoprotein B antibody coated in ELISA plates. LDL bound E2 protein was detected with a monoclonal anti-E2 antibody, followed by an alkaline-phosphatase labeled secondary antibody and PNPP substrate. The subtracted blank consisted of E2 protein in the absence of LDL.

FIG. 3A; FIG. 3B; and FIG. 3C: Incubation of LDL enhanced binding of HCV E2. Preincubation of recombinant HCV E2 protein with human LDL results in increased binding of E2 protein to MOLT-4 cells (FIG. 3A and FIG. 3B) and Huh-7 cells (FIG. 3C) at 4° C. Cell-bound E2 was detected using a monoclonal antiE2 antibody (FIG. 3A and FIG. 3C) or a polyclonal anti-E2 serum (FIG. 3B). Preincubation with human LDL did not increase binding of FITC-labeled recombinant HIV gp120 protein to MOLT-4 cells (FIG. 3A).

FIG. 3D: Preincubation with E2 enhanced binding of labeled LDL. Preincubation of human LDL with recombinant HCV E2 protein resulted in increased binding of LDL to MOLT-4 cells at 4° C. FITC-labeled LDL at concentrations of 1, 5 and 10 μg/ml was incubated with HCV E2 protein and the amount of cell-bound fluorescent LDL was determined by Flow cytometry.

FIG. 3E: Enhanced LDL binding demonstrated by both labeled LDL and using goat anti-LDL.

FIG. 3F: Mouse anti-LDL does not detect E2-LDL binding (confirming earlier data).

FIG. 4A: Expression of human CD81 on parental mouse 3T3 cells and 3T3 cells transfected with human CD81. Cells were detached using a non-enzymatic cell-dissociation solution (Sigma) and stained with anti-CD81 mononclonal antibody JS46, followed by an fluorescent-labeled anti-mouse IgG secondary antibody. Cells were analyzed by flow cytometry.

FIG. 4B: Binding of recombinant HCV E2 to mouse 3T3 cells. Cells were detached non-enzymatically and incubated with recombinant E2 protein. Cell-bound E2 was detected with monoclonal anti-E2 antibody 108, followed by incubation with a secondary fluorescent labeled anti-human IgG. Cells were analyzed by flow cytometry.

FIG. 4C: Binding of HCV E2 and HCV-E2/LDL to mouse 3T3-human CD81 cells. Cells were detached non-enzymatically and incubated with E2 alone or E2/LDL. Cell-bound E2 was detected with monoclonal anti-E2 antibody 108, followed by incubation with a secondary fluorescent labeled anti-human IgG. Cells were analyzed by flow cytometry.

FIG. 4D: Binding of recombinant HCV E2 and E2-LDL to human fibroblasts (FSF) and human fibroblasts negative for the human LDLr (Null). Cells were detached non-enzymatically and incubated with E2 or E2/LDL proteins. Cell-bound E2 was detected with monoclonal anti-E2 antibody 108, followed by incubation with a secondary fluorescent labeled anti-human IgG. Cells were analyzed by flow cytometry.

FIG. 4E and FIG. 4F: HCV-E2 does not bind to apoprotein B. MOLT-4 cells were incubated with E2 protein, apo-B delipidized protein and E2/apoB. Cell-bound apo-B was detected using a goat anti-human apoprotein B polyclonal antibody followed by a fluorescent labeled anti-goat IgG (A). Cell-bound E2 was detected with monoclonal anti-E2 antibody 108, followed by incubation with a secondary fluorescent labeled anti-human IgG. Cells were analyzed by flow cytometry.

FIG. 4G and FIG. 4H: HCV-E2 interacts with human and bovine lipoproteins. MOLT-4 cells were incubated with E2 protein alone or E2 protein with human VLDL, LDL or HDL (FIG. 4G) or bovine lipoprotein (FIG. 4H). Controls consisted of cells incubated with the individual lipoproteins alone. Cell-bound E2 was detected with monoclonal anti-E2 antibody 108, followed by incubation with a secondary fluorescent labeled anti-human IgG. Cells were analyzed by flow cytometry.

FIG. 5A: Regulation of human LDLr expression by bovine lipoproteins. MOLT-4 cells were grown in RPMI medium supplemented with 10% fetal calf serum (FCS) or in medium supplemented with lipoprotein deficient FCS (LPDFCS). Cells were stained with a mouse monoclonal antibody against LDLr, followed by a fluorescent labeled goat anti-mouse IgG. Cells were analyzed in a Flow cytometer.

FIG. 5B: MOLT 4 cells—D×S and rescue.

FIG. 5C: Binding of labeled LDL after removal of bovine lipoproteins with dextrane sulfate.

FIG. 6: HCV-E2 associates with LDL and binds as complex to both hCD81 and hLDLr.

### DESCRIPTION OF ILLUSTRATIVE EMBODIMENTS

Coronary heart disease is a leading cause of mortality, and the associated healthcare costs are staggering. Atherosclerosis is a leading contributor to this disease, which in turn is closely related to the presence of elevated cholesterol in the bloodstream of affected individuals. While diet and drug therapy can address hypercholesteremia to an extent, both of these approaches may be self-limiting. As such, there is an urgent need to find new ways to address this significant health risk.

The viral genomic sequence of HCV is known, as are methods for obtaining the sequence. See, International Publication Nos. WO 89/04669; WO 90/11089; and WO 90/14436. Hepatitis C Virus (HCV) HCV is an enveloped virus containing a positive-sense single-stranded RNA genome of approximately 9.5 kb. The genomic sequence of HCV is approximately 9401 base pairs in length (SEQ. ID. NO: 1). The peptide sequence for HCV can be obtained from Genbank Accession No. M62321 (SEQ ID NO: 2). The viral genome consists of a lengthy 5′ untranslated region (UTR), a long open reading frame encoding a polyprotein precursor of approximately 3011 amino acids (SEQ ID NO: 2) and a short 3′ UTR. The 5′ UTR is the most highly conserved part of the HCV genome and is important for the initiation and control of polyprotein translation. Translation of the HCV genome is initiated by a cap-independent mechanism known as internal ribosome entry. This mechanism involves the binding of ribosomes to an RNA sequence known as the internal ribosome entry site (IRES). The polyprotein precursor is cleaved by both host and viral proteases to yield mature viral structural and non-structural proteins. Viral structural proteins include a nucleocapsid core protein and two envelope glycoproteins, E1 and E2 (U.S. Pat. No. 6,326,151).

HCV utilizes the low density lipoprotein receptor (LDLr) for cell binding and entry (Wuenschmann et al., 2000; Monazahian et al., 1999; Agello et al., 1999). The inventors have discovered that the HCV envelope glycoprotein (HCV E2 glycoprotein) binds to the lipid moiety of human lipoproteins, and the lipid-virus complex uses the natural receptor for LDL to bind to cells. The HCV E2 glycoprotein starts at amino acid 394 of the HCV polyprotein, and extends to amino acid 747. It has a hypervariable region at the amino terminus of the protein, and the carboxy terminus includes a transmembrane domain. HCV enters the cell via endocytosis using the LDL receptor. HCV E2 glycoprotein interactions with LDL result not only in CD81-independent binding to cells (Wuenschmann et al., 2000), but also to enhancement in LDL binding and uptake by the cells. Thus, the present inventors have identified a novel mechanism by which HCV gains entry into a target cell, thereby providing for therapeutic intervention. Moreover, the inventors now disclose the use of the HCV E2 glycoprotein to lower blood cholesterol levels in a subject. In other embodiments, the inventors further contemplate new and useful methods for identifying inhibitors of HCV infection and agents that reduce hypercholesteremia. These and other aspects of the invention are described in greater detail below.

A. HCV E2 Glycoprotein and Peptides Thereof

In various aspects of the invention, applicants envision the use of both full length E2 glycoprotein and peptides thereof. The following is a discussion of methods of making and using these compositions.

1. Peptide Synthesis and Chemical Degradation

The peptides of the invention can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), Houghten et al. (1985). In some embodiments, peptide synthesis is contemplated by using automated peptide synthesis machines, such as those available from Applied Biosystems (Foster City, Calif.). The peptides of the present invention may be isolated and extensively dialyzed to remove undesired small molecular weight molecules and/or lyophilized for more ready formulation into a desired vehicle. Short peptide sequences, or libraries of overlapping peptides, can be readily synthesized and then screened in screening assays designed to identify reactive peptides. Peptides with at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50, 55, 60, 65, 70, 75, 80, 85, 90, 95 or up to about 100 amino acid residues are contemplated by the present invention.

Longer peptides or polypeptides also may be prepared by recombinant means, e.g., by the expression of a nucleic acid sequence encoding a peptide or polypeptide comprising an HCV E2 glycoprotein or peptides thereof in vitro translation system or in a living cell, as described in detail below. By way of example only, in certain embodiments, a nucleic acid encoding an HCV E2 glycoprotein or peptides thereof is comprised in, for example, a vector in a recombinant cell. The nucleic acid may be expressed to produce an HCV E2 glycoprotein or peptides thereof. The HCV E2 glycoprotein or peptides thereof may be secreted from the cell, or comprised as part of or within the cell.

In other embodiments, HCV E2 peptides may be produced by chemical degradation of an HCV E2 glycoprotein. For example, protease digestion.

2. Purification of Proteins

In certain aspects of the invention, purification of HCV E2 glycoprotein or peptides thereof will be desired. The term “purified proteins, polypeptides, or peptides” as used herein, is intended to refer to a proteinaceous composition, isolatable from mammalian cells or recombinant host cells, wherein the at least one protein, polypeptide, or peptide is purified to any degree relative to its naturally-obtainable state, i.e., relative to its purity within a cellular extract. A purified protein, polypeptide, or peptide therefore also refers to a wild-type or mutant protein, polypeptide, or peptide free from the environment in which it naturally occurs.

Generally, “purified” will refer to a specific protein, polypeptide, or peptide composition that has been subjected to fractionation to remove various other proteins, polypeptides, or peptides, and which composition substantially retains its activity, as may be assessed, for example, by the protein assays, as described herein below, or as would be known to one of ordinary skill in the art for the desired protein, polypeptide or peptide.

Where the term “substantially purified” is used, this will refer to a composition in which the specific protein, polypeptide, or peptide forms the major component of the composition, such as constituting about 50% of the proteins in the composition or more. In preferred embodiments, a substantially purified protein will constitute more than 60%, 70%, 80%, 90%, 95%, 99% or even more of the proteins in the composition.

A peptide, polypeptide or protein that is “purified to homogeneity,” as applied to the present invention, means that the peptide, polypeptide or protein has a level of purity where the peptide, polypeptide or protein is substantially free from other proteins and biological components. For example, a purified peptide, polypeptide or protein will often be sufficiently free of other protein components so that degradative sequencing may be performed successfully.

Various methods for quantifying the degree of purification of proteins, polypeptides, or peptides will be known to those of skill in the art in light of the present disclosure. These include, for example, determining the specific protein activity of a fraction, or assessing the number of polypeptides within a fraction by gel electrophoresis.

To purify a desired protein, polypeptide, or peptide a natural or recombinant composition comprising at least some specific proteins, polypeptides, or peptides will be subjected to fractionation to remove various other components from the composition. In addition to those techniques described in detail herein below, various other techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite, lectin affinity, immunoaffinity chromatography and other affinity chromatography steps; isoelectric focusing; gel electrophoresis, HPLC; and combinations of such and other techniques.

In certain embodiments, the protein or peptides of the present invention may be purified by High Performance Liquid Chromatography (HPLC). HPLC is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

In other embodiments, Gel chromatography, or molecular sieve chromatography may be used. Gel chromatography is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

In other embodiments, Affinity Chromatography may be used. Affinity chromotography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (e.g., alter pH, ionic strength, and temperature.).

Although preferred for use in certain embodiments, there is no general requirement that the protein, polypeptide, or peptide always be provided in their most purified state. Indeed, it is contemplated that less substantially purified protein, polypeptide or peptide, which are nonetheless enriched in the desired protein compositions, relative to the natural state, will have utility in certain embodiments.

Methods exhibiting a lower degree of relative purification may have advantages in total recovery of protein product, or in maintaining the activity of an expressed protein. Inactive products also have utility in certain embodiments, such as, e.g., in determining antigenicity via antibody generation.

In another embodiment of the present invention, the inventors have contemplated methods of removing plasma lipoproteins from a blood sample. This aspect of the present invention may be used, for example, in determining the amount of LDL levels in a subjects blood sample. In removing the plasma lipoproteins, the inventors contemplate the use of isolated HCV E2 glycoprotein attached to a support. In certain aspects of this invention, the support may be a non-reactive solid support. By way of example only, it is contemplated that nitrocellulose membrane, bead, glass supports or any other conventional methods may be used.

B. Nucleic Acids and Expression of HCV E2 Glycoprotein

1. Nucleic Acids

A nucleic acid may be made by any technique known to one of ordinary skill in the art, such as for example, chemical synthesis, enzymatic production or biological production. In one embodiment, the inventors contemplate producing HCV E2 glycoprotein fragments thereof for use in the present invention using recombinant expression. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266 032, or via deoxynucleoside H-phosphonate intermediates as described by U.S. Pat. No. 5,705,629. Also, various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244.

A non-limiting example of an enzymatically produced nucleic acid include one produced by enzymes in amplification reactions such as PCR™ (see for example, U.S. Pat. No. 4,683,202 and U.S. Pat. No. 4,682,195), or the synthesis of an oligonucleotide described in U.S. Pat. No. 5,645,897. A non-limiting example of a biologically produced nucleic acid includes a recombinant nucleic acid produced (i.e., replicated) in a living cell, such as a recombinant DNA vector replicated in bacteria (see for example, Sambrook et al. 1989).

The nucleic acid segments of the present invention include those encoding functionally equivalent HCV E2 glycoproteins, as described above. Such sequences may arise as a consequence of codon redundancy (Table 1) and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function.

TABLE 1

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

2. Nucleic Acid Segments

In certain embodiments, the nucleic acid is a nucleic acid segment. As used herein, the term “nucleic acid segment,” are smaller fragments of a nucleic acid, such as for non-limiting example, those that encode only part of the peptide or polypeptide sequence. Thus, a “nucleic acid segment” may comprise any part of a gene sequence, of from about 2 nucleotides to the full length of the peptide or polypeptide encoding region.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

3. Purification of Nucleic Acids

A nucleic acid may be purified on polyacrylamide gels, cesium chloride centrifugation gradients, or by any other means known to one of ordinary skill in the art (see for example, Sambrook et al., 1989).

In a certain aspect, the present invention concerns a nucleic acid that encodes an HCV E2 glycoprotein or peptides thereof. The nucleic acids may be an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

4. Expression Vectors and Systems

In another aspect of the present invention, it is contemplated that expression vectors may be used to express HCV E2 glycoprotein or peptides thereof. It is also contemplated that expression vectors may be used to deliver nucleic acid segments encoding HCV E2 glycoprotein or peptides thereof to a target cell. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

5. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., in frame of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the -lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, circulatory system, or organism chosen for expression. Those of skill in the art of molecular biology generally know the use of promoters, enhancers, and cell type combinations for protein expression, (see, for example Sambrook et al. 1989). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Table 2 lists non-limiting examples of elements/promoters that may be employed, in the context of the present invention, to regulate the expression of a RNA. Table 3 provides non-limiting examples of inducible elements, which are regions of a nucleic acid sequence that can be activated in response to a specific stimulus.

TABLE 2
Promoter and/or Enhancer

| Promoter/Enhancer | References |
|---|---|
| Immunoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton |
| | et al.; 1990 |
| Immunoglobulin Light Chain | Queen et al., 1983; Picard et al., 1984 |
| T-Cell Receptor | Luria et al., 1987; Winoto et al., 1989; Redondo et al.; 1990 |
| HLA DQ a and/or DQ | Sullivan et al., 1987 |
| Interferon | Goodbourn et al., 1986; Fujita et al., 1987; Goodbourn |
| | et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| Actin | Kawamoto et al., 1988; Ng et al.; 1989 |
| Muscle Creatine Kinase (MCK) | Jaynes et al., 1988; Horlick et al., 1989; Johnson et al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| Fetoprotein | Godbout et al., 1988; Campere et al., 1989 |
| Globin | Bodine et al., 1987; Perez-Stable et al., 1990 |
| Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsch et al., 1990 |
| (NCAM) | |
| 1-Antitrypsin | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A | Edbrooke et al., 1989 |
| (SAA) | |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., 1985; |
| | Firak et al., 1986; Herr et al., 1986; Imbra et al., 1986; |
| | Kadesch et al., 1986; Wang et al., 1986; Ondek et al., 1987; |
| | Kuhl et al., 1987; Schaffner et al., 1988 |
| Polyoma | Swartzendruber et al., 1975; Vasseur et al., 1980; Katinka |
| | et al., 1980, 1981; Tyndell et al., 1981; Dandolo et al., 1983; |
| | de Villiers et al., 1984; Hen et al., 1986; Satake et al., 1988; |
| | Campbell and/or Villarreal, 1988 |
| Retroviruses | Kriegler et al., 1982, 1983; Levinson et al., 1982; Kriegler |
| | et al., 1983, 1984a, b, 1988; Bosze et al., 1986; Miksicek |
| | et al., 1986; Celander et al., 1987; Thiesen et al., 1988; |
| | Celander et al., 1988; Choi et al., 1988; Reisman et al., 1989 |
| Papilloma Virus | Campo et al., 1983; Lusky et al., 1983; Spandidos and/or |
| | Wilkie, 1983; Spalholz et al., 1985; Lusky et al., 1986; |
| | Cripe et al., 1987; Gloss et al., 1987; Hirochika et al., 1987; |
| | Stephens et al., 1987 |
| Hepatitis B Virus | Bulla et al., 1986; Jameel et al., 1986; Shaul et al., 1987; |
| | Spandau et al., 1988; Vannice et al., 1988 |
| Human Immunodeficiency | Muesing et al., 1987; Hauber et al., 1988; Jakobovits et al., |
| Virus | 1988; Feng et al., 1988; Takebe et al., 1988; Rosen et al., |
| | 1988; Berkhout et al., 1989; Laspia et al., 1989; Sharp et al., |
| | 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 3
Inducible Elements

| Element | Inducer | References |
|---|---|---|
| MT II | Phorbol Ester (TFA) | Palmiter et al., 1982; Haslinger |
| | Heavy metals | et al., 1985; Searle et al., 1985; |
| | | Stuart et al., 1985; Imagawa et al., |
| | | 1987, Karin et al., 1987; Angel |
| | | et al., 1987b; McNeall et al., 1989 |
| MMTV (mouse mammary | Glucocorticoids | Huang et al., 1981; Lee et al., |
| tumor virus) | | 1981; Majors et al., 1983; Chandler |
| | | et al., 1983; Lee et al., 1984; Ponta |
| | | et al., 1985; Sakai et al., 1988 |
| Interferon | Poly(rI)x | Tavernier et al., 1983 |
| | Poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle Disease | Hug et al., 1988 |
| | Virus | |
| GRP78 Gene | A23187 | Resendez et al., 1988 |
| 2-Macroglobulin | IL-6 | Kunz et al., 1989 |
| Vimentin | Serum | Rittling et al., 1989 |
| MHC Class I Gene H-2b | Interferon | Blanar et al., 1989 |
| HSP70 | E1A, SV40 Large T Antigen | Taylor et al., 1989, 1990a, 1990b |
| Proliferin | Phorbol Ester-TPA | Mordacg et al., 1989 |
| Tumor Necrosis Factor | PMA | Hensel et al., 1989 |
| Thyroid Stimulating | Thyroid Hormone | Chatterjee et al., 1989 |
| Hormone Gene | | |

The identity of tissue-specific promoters or elements, as well as assays to characterize their activity, is well known to those of skill in the art. Non limiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

6. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819).

7. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997). “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

8. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997).

9. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

10. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

11. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

12. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

13. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

14. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Non-limiting examples of virus vectors that may be used to deliver a nucleic acid of the present invention are described below.

i. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

ii. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368.

iii. Retroviral Vectors

Retroviruses have promise for delivering nucleic acid sequences encoding HCV E2 glycoproteins to a subject due to their ability to integrate their genes into the host genome, transferring a large amount of foreign genetic material, infecting a broad spectrum of species and cell types and of being packaged in special cell-lines (Miller, 1992).

In order to construct retroviral vector of the present invention, a nucleic acid (e.g., one encoding an HCV E2 glycoprotein) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

iv. Other Viral Vectors

Other viral vectors may be employed for delivering a nucleic acid encoding an HCV E2 glycoprotein to a subject. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

15. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al., 1989), by injection (U.S. Pat. Nos. 5,994,624, 5,981,274, 5,945,100, 5,780,448, 5,736,524, 5,702,932, 5,656,610, 5,589,466 and 5,580,859), including microinjection (Harlan and Weintraub, 1985; U.S. Pat. No. 5,789,215); by electroporation (U.S. Pat. No. 5,384,253; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988). Through the application of techniques such as these, organelle(s), cell(s), tissue(s) or organism(s) may be stably or transiently transformed.

16. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

A tissue may comprise a host cell or cells to be transformed with a nucleic acid encoding an HCV E2 glycoprotein. In other embodiments, the nucleic acid may encode a peptide, protein, or polypeptide that inhibits or reduces HCV E2 glycoprotein binding to plasma lipoprotein. In yet other embodiments, the nucleic acid may encode a peptide or protein that inhibits or reduces HCV E2 glycoprotein/plasma lipoprotein complex binding to LDL receptor. The tissue may be part or separated from an organism. In certain embodiments, a tissue may comprise, but is not limited to, adipocytes, alveolar, ameloblasts, axon, basal cells, blood (e.g., lymphocytes), blood vessel, bone, bone marrow, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, skin, small intestine, spleen, stem cells, stomach, testes, anthers, ascite tissue, cobs, ears, flowers, husks, kernels, leaves, meristematic cells, pollen, root tips, roots, silk, stalks, and all cancers thereof.

In certain embodiments, the host cell or tissue may be comprised in at least one organism. In certain embodiments, the organism may be, but is not limited to, a prokayote (e.g., a eubacteria, an archaea) or an eukaryote, as would be understood by one of ordinary skill in the art (see, for example, phylogeny.arizona.edu/tree/phylogeny.html).

Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5, JM109, and KC8, bacilli such as Bacillus subtilis; and other enterobacteriaceae such as Salmonella typhimurium, Serratia marcescens, various Pseudomonas specie, as well as a number of commercially available bacterial hosts such as SURE® Competent Cells and SOLOPACK Gold Cells (STRATAGENE®, La Jolla). In certain embodiments, bacterial cells such as E. coli LE392 are particularly contemplated as host cells for phage viruses.

Examples of eukaryotic host cells for replication and/or expression of a vector include, but are not limited to, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

It is contemplated that the proteins, polypeptides or peptides produced by the methods of the invention may be “overexpressed”, i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell and, e.g., visible on a gel.

D. Screening Assays

One important aspect of the present invention concerns assays for screening for potential inhibitors of HCV infection. In other embodiments, it is contemplated that screening assays may be used to identify particular HCV E2 peptides that may be effective in lowering LDL levels in a subject. The assays may be carried out at the protein or nucleic acid level. Such assays may find use in diagnostic applications for directing the treatment of a subject infected with HCV or of a subject with elevated LDL levels. The assays may even provide insight as to the relative efficacy of a given inhibitor of HCV infection or of a particular HCV E2 peptide.

The present invention provides methods for screening for novel inhibitors that reduce E2 glycoprotein binding to plasma lipoprotein. In other embodiments, the inhibitors may reduce E2 glycoprotein/plasma lipoprotein complex binding to an LDL receptor. In yet other embodiments, methods for screening for E2 peptides that may be effective in lowering LDL levels in a patient are disclosed.

1. Assay Formats to Screen for Inhibitors

In certain embodiments, the present invention provides methods for screening and identifying candidate substances that inhibits HCV infection of a target cell. In other embodiments, it is contemplated that screening assays may be used to identify particular HCV E2 peptides that may be effective in lowering LDL levels in a subject. A candidate substance that reduces E2 glycoprotein binding to plasma lipoprotein or E2 glycoprotein/plasma lipoprotein complex binding to a low density lipoprotein receptor (LDLr) can inhibit HCV infection of a target cell. This may be achieved by obtaining target amino acid sites on either the E2 glycoprotein, plasma lipoprotein, or LDLr, and contacting the amino acid site with candidate substances followed by assays to determine the formation of either an E2 glycoprotein/plasma lipoprotein complex or an E2 glycoprotein/plasma lipoprotein/LDLr complex. Alternatively, an E2 glycoprotein may be admixed with a plasma lipoprotein and a candidate substance under conditions effective to allow the formation of an E2 glycoprotein/plasma lipoprotein complex followed by assays to determine the reduction in E2 glycoprotein binding to plasma lipoprotein, as compared to binding in the absence of the candidate substance. In yet another embodiment, an E2 glycoprotein/plasma lipoprotein complex may be admixed with a LDLr and a candidate substance under conditions effective to allow the formation of an E2 glycoprotein/plasma lipoprotein/LDLr complex followed by assays to determine the reduction in E2 glycoprotein/plasma lipoprotein complex binding to LDLr, as compared to binding in the absence of the candidate substance. In other embodiments, E2 peptides may be screened to determine their efficacy in binding to LDL and subsequent internalization of the E2 peptide/LDL complex.

Candidate substances can include fragments or parts of naturally-occurring compounds or may be only found as active combinations of known compounds which are otherwise inactive. In one embodiment, the candidate substances are small molecules. In yet other embodiments, candidate substances may be synthetic or natural E2 peptides. Alternatively, it is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

i. In Vitro Assays

A straightforward assay to run is a binding assay. Binding of a molecule to a target may, in and of itself, be inhibitory, due to steric, allosteric or charge-charge interactions. This can be performed in solution or on a solid phase and can be utilized as a first round screen to rapidly eliminate certain compounds before moving into more sophisticated screening assays. The target may be either free in solution, fixed to a support, expressed in or on the surface of a cell. Examples of supports include nitrocellulose, a column or a gel. Either the target or the compound may be labeled, thereby permitting determining of binding. In another embodiment, the assay may measure the enhancement of binding of a target to a natural or artificial substrate or binding partner. Usually, the target will be the labeled species, decreasing the chance that the labeling will interfere with the binding moiety's function. One may measure the amount of free label versus bound label to determine binding or inhibition of binding. In other embodiments, binding of E2 glycoprotein to plasma lipoprotein, or binding of E2 glycoprotein/plasma lipoprotein complex to LDLr may be determined by gel electrophoresis, gel filtration chromatography, fluorescence quenching, flow cytometry, elisa, solid phase immunoassay, or confocal microscopy.

A technique for high throughput screening of compounds is described in PCT Application WO 84/03564. In high throughput screening, large numbers of candidate inhibitory test compounds, which may be small molecules, natural substrates and ligands, or may be fragments or structural or functional mimetics thereof, are synthesized on a solid substrate, such as plastic pins or some other surface. Alternatively, purified target molecules can be coated directly onto plates or supports for use in drug screening techniques. Also, fusion proteins containing a reactive region (preferably a terminal region) may be used to link an active region of an enzyme to a solid phase, or support. The test compounds are reacted with the target molecule, and bound test compound is detected by various methods (see, e.g., Coligan et al. (1991)).

Examples of small molecules that may be screened include, but are not limited to, small organic molecules, peptides or peptide-like molecules, nucleic acids, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays of the invention to identify compounds that inhibit or reduce the binding of HCV E2 glycoprotein to plasma lipoprotein or E2 glycoprotein/plasma lipoprotein complex to LDLr. Further, in drug discovery, for example, proteins have been fused with antibody Fc portions for the purpose of high-throughput screening assays to identify potential modulators of new polypeptide targets. See, Bennett et al., (1995) and Johanson et al., (1995).

ii. In Vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more HCV characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

In vivo assays generally include the steps of: administering a candidate substance to a subject; and determining the ability of the candidate substance to reduce or prevent one or more characteristics of the infection HCV. In other embodiments, administration of a candidate E2 peptide to a subject; and determining the ability of the candidate substance to reduce LDL levels in the subject.

Treatment of these animals with candidate substances will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

iii. Arrays

Hi-throughput assays, for example, arrays comprising a plurality of ligands arranged on a solid support, represent an important diagnostic tool provided by the invention. The use of arrays involves the placement and binding of nucleic acids, or another type of ligand having affinity for a molecule in a test sample, to known locations, termed sectors, on a solid support.

Arrays can be used, through hybridization of a test sample to the array, to determine the presence or absence of a given molecule in the sample. By including any additional other target nucleic acids or other types of ligands, potentially thousands of target molecules can be simultaneously screened for in a test sample. Many different methods for preparation of arrays comprising target substances arranged on solid supports are known to those of skill in the art and could be used in accordance with the invention. Specific methods for preparation of such arrays are disclosed in, for example, Affinity Techniques, Enzyme Purification: Jakoby and Wilchek, (1974) and Dunlap, (1974). Examples of other techniques which have been described for the attachment of test materials to arrays include the use of successive application of multiple layers of biotin, avidin, and extenders (U.S. Pat. No. 4,282,287); methods employing a photochemically active reagent and a coupling agent which attaches the photoreagent to the substrate (U.S. Pat. No. 4,542,102); use of polyacrylamide supports on which are immobilized oligonucleotides (PCT Patent Publication 90/07582); use of solid supports on which oligonucleotides are immobilized via a 5′-dithio linkage (PCT Patent Publication 91/00868); and through use of a photoactivateable derivative of biotin as the agent for immobilizing a biological polymer of interest onto a solid support (see U.S. Pat. No. 5,252,743; and PCT Patent Publication 91/07087). In the case of a solid support made of nitrocellulose or the like, standard techniques for UV-crosslinking may be of particular utility (Sambrook et al., 1989).

The solid support surface upon which an array is produced in accordance with the invention may potentially be any suitable substance. Examples of materials which may be used include polymers, plastics, resins, polysaccharides, silica or silica-based materials, carbon, metals, inorganic glasses, membranes, etc. It may also be advantageous to use a surface which is optically transparent, such as flat glass or a thin layer of single-crystal silicon. Contemplated as being especially useful are nylon filters, such as Hybond N+ (Amersham Corporation, Amersham, UK). Surfaces on the solid substrate will usually, though not always, be composed of the same material as the substrate, and the surface may further contain reactive groups, which could be carboxyl, amino, hydroxyl, or the like.

It is contemplated that one may wish to use a solid support surface which is provided with a layer of crosslinking groups (U.S. Pat. No. 5,412,087). Crosslinking groups could be selected from any suitable class of compounds, for example, aryl acetylenes, ethylene glycol oligomers containing 2 to 10 monomer units, diamines, diacids, amino acids, or combinations thereof. Crosslinking groups can be attached to the surface by a variety of methods that will be readily apparent to one of skill in the art. For example, crosslinking groups may be attached to the surface by siloxane bonds formed via reactions of crosslinking groups bearing trichlorosilyl or trisalkoxy groups with hydroxyl groups on the surface of the substrate. The crosslinking groups can be attached in an ordered array, i.e., as parts of the head groups in a polymerized Langmuir Blodgett film. The linking groups may be attached by a variety of methods that are readily apparent to one skilled in the art, for instance, by esterification or amidation reactions of an activated ester of the linking group with a reactive hydroxyl or amine on the free end of the crosslinking group.

A significant benefit of the arrays of the invention is that they may be used to simultaneously screen for inhibitors that inhibit the formation of an HCV E2 glycoprotein/plasma lipoprotein complex or an E2 glycoprotein/plasma lipoprotein/LDLr complex. Use of the arrays generally will comprise, in a first step, contacting the array with a test sample. Generally the test sample will be labeled to facilitate detection of hybridizing test samples. By detection of test samples having affinity for bound target nucleic acids or other ligands, the identity of the target molecule will be known.

Following contacting with the test sample, the solid support surface is then generally washed free of unbound test sample, and the signal corresponding to the probe label is identified for those regions on the surface where the test sample has high affinity. Suitable labels for the test sample include, but are not limited to, radiolabels, chromophores, fluorophores, chemiluminescent moieties, antigens and transition metals. In the case of a fluorescent label, detection can be accomplished with a charge-coupled device (CCD), fluorescence microscopy, or laser scanning (U.S. Pat. No. 5,445,934). When autoradiography is the detection method used, the marker is a radioactive label, such as ³²P, and the surface is exposed to X-ray film, which is developed and read out on a scanner or, alternatively, simply scored manually. With radiolabeled probes, exposure time will typically range from one hour to several days. Fluorescence detection using a fluorophore label, such as fluorescein, attached to the ligand will usually require shorter exposure times. Alternatively, the presence of a bound probe may be detected using a variety of other techniques, such as an assay with a labeled enzyme, antibody, or the like. Detection also may, in the case of nucleic acids, alternatively be carried out using PCR. In this instance, PCR detection may be carried out in situ on the slide. In this case one may wish to utilize one or more labeled nucleotides in the PCR mix to produce a detectable signal. Other techniques using various marker systems for detecting bound ligand will also be readily apparent to those skilled in the art.

2. Antibodies

In certain embodiment, the present invention will examine the ability of various E2 or E2-binding peptides to interact with E2 or the LDL-receptor. Peptides are discussed elsewhere in this document. In another aspect, the present invention contemplates use of an anti-E2 glycoprotein antibody that is immunoreactive with HCV E2 glycoprotein in preventing or reducing the formation of an E2 glycoprotein/plasma lipoprotein complex. In another embodiments, use of an anti-LDL receptor antibody which is immunoreactive to an LDL receptor is contemplated in preventing the formation of an E2 glycoprotein/plasma lipoprotein/LDL receptor complex. In still another embodiments of the present invention, use of an anti-E2 glycoprotein/plasma lipoprotein antibody which is immunoreactive with an E2 glycoprotein/plasma lipoprotein complex is contemplated in preventing the formation of an E2 glycoprotein/plasma lipoprotein/LDL receptor complex.

An antibody can be a polyclonal or a monoclonal antibody. In a preferred embodiment, an antibody is a monoclonal antibody. Means for preparing and characterizing antibodies are well known in the art (see, e.g., Harlow and Lane, 1988)

Briefly, a polyclonal antibody is prepared by immunizing an animal with an immunogen comprising a polypeptide and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265. Typically, this technique involves immunizing a suitable animal with a selected immunogen composition, polypeptide or peptide or cell expressing high levels of an immunogen. The immunizing composition is administered in a manner effective to stimulate antibody producing cells. Rodents such as mice and rats are preferred animals, however, the use of rabbit, sheep frog cells is also possible. The use of rats may provide certain advantages (Goding, (1986)), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984; Burden and Von Knippenberg (1984). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

E. Formulations and Routes for Administration to Subjects

Where clinical applications are contemplated, it will be necessary to prepare pharmaceutical compositions of the HCV E2 glycoprotein, HCV E2 peptides or other agents in a form appropriate for the intended application.

Pharmaceutical compositions of the present invention comprise an effective amount of one or more compounds (e.g. inhibitors of HCV infection) dissolved or dispersed in a pharmaceutically acceptable carrier. In another embodiment, the pharmaceutical composition comprises an effective amount of an HCV E2 glycoprotein or peptide thereof. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains an HCV E2 glycoprotein, HCV E2 peptide, inhibitor of HCV infection, or any other compound contemplated by the present disclosure will be known to those of skill in the art in light of the present disclosure, as exemplified by Remington's Pharmaceutical Sciences, 18th Ed. Mack Printing Company, 1990. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's, 1990). Except insofar as any conventional carrier is incompatible with the active ingredient, its use in the therapeutic or pharmaceutical compositions is contemplated.

The active compositions of the present invention may include classic pharmaceutical preparations. Administration of these compositions according to the present invention will be via any common route so long as the target tissue is available via that route. Although, the intravenous route is a preferred embodiment, other routes of administration are contemplated. This includes, intradermally, intraarterially, intraperitoneally, intralesionally, intracranially, intraarticularly, intraprostaticaly, intrapleurally, intratracheally, intranasally, intravitreally, intravaginally, intrarectally, topically, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (Remington's, 1990).

The active compounds also may be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

An HCV E2 glycoprotein, E2 peptide, or the inhibitors of HCV E2 glycoprotein/plasma lipoprotein complex formation or E2 glycoprotein/plasma lipoprotein/LDLr complex formation may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the HCV E2 glycoprotein, E2 peptide, or the inhibitors of HCV E2 glycoprotein/plasma lipoprotein complex formation or E2 glycoprotein/plasma lipoprotein/LDLr complex formation are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof, a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

F. Combination Therapy

In order to increase the effectiveness of a full-length, substantially full-length, or truncated HCV E2 glycoprotein, HCV E2 peptide, or expression construct coding therefore, it may be desirable to combine these compositions with other agents effective in lowering LDL levels in a subject. Examples of other agents effective in lowering LDL levels in a subject include nicotinic acid, clofibrate, dextrothyroxine sodium, neomycin, beta-sitosterol, probucol, cholestyramine, the “statin” class of drugs (for example, cerivastatin, fluvastatin, atorvastatin, lovastatin, pravastatin, and simvastatin) also known as HMG-CoA reductase inhibitors.

In other embodiments, in order to increase the effectiveness of inhibitors of HCV infection (e.g., inhibitors of HCV E2 glycoprotein binding to plasma lipoprotein or inhibitors of HCV E2 glycoprotein/plasma lipoprotein complex binding to LDL receptor) or expression constructs coding therefore, it may be desirable to combine these compositions with other agents effective in inhibiting HCV infection in a subject. Examples of other agents effective in inhibiting HCV infection in a subject include alpha interferon, ribavirin, or Peginterferon Alfa-2b. Potential treatments include drugs that inhibit viral uncoating (e.g., amantidine), and inhibitors of HCV replication enzymes (e.g., helicase inhibitors, polymerase inhibitors, protease inhibitors, etc.).

More generally, these other compositions would be provided in a combined amount effective to lower LDL levels in a subject or inhibit HCV infection in a subject. This process may involve contacting the blood stream or target cell with the expression construct and the agent(s) or multiple factor(s) at the same time. This may be achieved by contacting the blood stream or target cell with a single composition or pharmacological formulation that includes both agents, or by contacting the cell with two distinct compositions or formulations, at the same time, wherein one composition includes the expression construct and the other includes the second agent(s).

Alternatively, the gene therapy may precede or follow the aforementioned treatments by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the bloodstream or target cell with both modalities within about 12-24 hours of each other and, more preferably, within about 6-12 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It is contemplated that various combinations of treatments for HCV infection may be employed. It is further contemplated that various combinations of treatments for lowering LDL levels in a subject may be employed. By way of example only, the following illustration is provided. For combination treatment of HCV infection, “A” may be gene therapy and “B” may be an agent or compound. In other aspects, “A” may be an agent or compound and “B” may also be another agent or compound. It is also contemplated that for treating elevated LDL levels in a subject, “A” may be gene therapy and “B” may be an agent or compound. In other aspects for treating elevated levels of LDL in a subject, “A” may be an agent or compound and “B” may also be another agent or compound.

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

G. Kits

In further embodiments, the invention provides HCV infection therapeutic kits. Such kits will generally comprise a pharmaceutically acceptable composition comprising an inhibitor of HCV infection. In yet other embodiments, the kits may include in combination with inhibitors of HCV infection, another agent effective in treating HCV infection.

In other embodiments, the invention provides kits for lowering LDL cholesterol levels in a subject. Such kits will generally comprise a pharmaceutically acceptable composition comprising an HCV E2 glycoprotein. In other embodiments, the HCV E2 glycoprotein will be substantially purified away from other HCV components. In yet other embodiments, the kits may include in combination with HCV E2 glycoprotein, another agent effective in lowering LDL levels in a subject.

The kits of the invention will generally comprise one or more containers into which the biological agents are placed and, preferably, suitably aliquoted. The components of the kits may be packaged either in aqueous media or in lyophilized form.

The container means of the kits will generally include at least one vial, test tube, flask, bottle, or even syringe or other container means, into which the peptide conjugated to the label may be placed, and preferably, suitably aliquoted. Where a second or third detectable label, binding ligand or additional component is provided, the kit will also generally contain a second, third or other additional container into which this label, ligand or component may be placed.

The kits of the present invention will also typically include a means for containing the inhibitors of HCV infection or the HCV E2 glycoprotein and any other reagent containers in close confinement for commercial sale. Such containers may include injection or blow-molded plastic containers into which the desired vials are retained.

### H. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### HCV and Mock Preparations

Plasma was obtained from patients with HCV-related chronic liver disease, or from HCV antibody negative and HCV RNA negative control subjects as previously described (Schmidt et al., 1995). HCV antibody and RNA testing was performed as previously described (Stapleton et al., 1999). HCV very low density (1.04-1.07 g/m³) and intermediate density particles (1.12-1.18 g/m³) were separated by sucrose gradient centrifugation as previously described (Wuenschmann et al., 2000; Xiang et al., 1998; Xiang et al., 1999). Negative control plasma (mock) were prepared in the same manner (Wuenschmann et al., 2000).

### Example 2

### Proteins and Antibodies

Purified recombinant HCV envelope glycoprotein E2 (Ala 384-Lys 715) and the non-structural proteins NS3/NS4 (Asp. 1569-Pro 1931) expressed in CHO cells were obtained from Austral Biologicals (San Remo, Calif.). HIV gp120-FITC was obtained from Bartels (Carlsbad, Calif.). Human and murine soluble CD81 was kindly provided by Dr. Shoshana Levy (Stanford University). Human LDL and labeled human LDL-BODIPY 488 was obtained from Molecular Probes (Eugene, Oreg.). Human VLDL, LDL and HDL were obtained from Biodesign (Saco, Me.). Bovine LDL was obtained from Sigma (St.Louis, Mo.). Anti-HCV E2 hMAb was used to detect HCV E2 bound to cells as previously described (Wunschmann et al., 2002). Anti-HIV gp120 hMAb F105 was kindly provided by Marshall Posner, Harvard Medical School. Polyclonal goat anti-LDL antibody was obtained from Sigma (St.Louis, Mo.). Normal goat serum (Sigma, St. Louis, Mo.) was used as isotype control. Anti-human LDL MAb (clone 4G3) was kindly provided by Dr. Jheem Medh (California State University, Northridge, Calif.)(35). Anti-human CD81 MAb (clone JS64) was obtained from RD Inc. (Flanders, N.J.). Nonspecific mouse IgG (Zymed; San Francisco, Calif.) was used as an isotype control. Species-specific fluorescent labeled secondary antibodies were obtained from Molecular Probes (Eugene, Oreg.).

### Example 3

### ELISA Assays

Microtiter plates were prepared by coating wells with 100 μl anti-LDL McAb (4 μg/ml in 100 mM bicarbonate buffer, pH 9.6) overnight (ON) at RT (Wuenschmann et al., 2000). Wells were washed with Tris-buffered saline (TBS; 150 mM NaCl, 20 mM Tris-HCl [pH 7.5]) and subsequently blocked by the addition of 150 μl of BLOTTO (TBS plus 0.1% Tween 20, 2.5% normal goat serum, and 2.5% nonfat dry milk) for 1 h at RT. Plates were washed twice with TBS followed by the addition of 100 μl of E2 protein or E2 protein with LDL in PBS. After ON incubation of samples at RT, plates were washed three times with TBS followed by the addition of anti HCV E2 McAb 108 (7.5 μg/ml). Plates were incubated for 1.5 h and washed three times with TBS; then 100 μl of anti-human IgG-alkaline phosphatase conjugate (Promega, Madison, Wis.) diluted 1/5,000 in BLOTTO was added for 1 h at RT washed four times with TBS, and incubated for 30 min with a 1-mg/ml solution of p-nitrophenyl phosphate (PNPP). Absorbance was measured at 405 nm with a multiwell plate reader.

### Example 4

### Cell Lines

MOLT-4 cells, a CD4⁺ T lymphoblastoid cell line, and Huh7 cells of hepatocyte origin were obtained from ATCC (Manassas, Va.). Human foreskin fibroblasts (FSF) and LDLr-deficient foreskin fibroblasts (Null) from a patient with familial hypercholesterolemia (Hobbs et al., 1987) were kindly provided by Dr. Jheem Medh.

MOLT-4 cells were cultured in RPMI 1640; whereas human fibroblasts and human Huh7 cells were propagated in DMEM. Media were supplemented with 10% fetal calf serum (FCS), 100 U/ml penicillin, 100 μg/ml streptomycin sulfate and 2 mM L-glutamine. Mouse 3T3 cells expressing human CD81 and control cells were kindly provided by Dr. Martin Hemler and Christopher Stipp (Dana Farber Cancer Center, Boston, Mass.) and were cultured in DMEM media, supplemented with 10% FCS, 100 U/ml penicillin, 100 μg/ml streptomycin sulfate, 2 mM L-glutamine and 0.1 mg/ml zeocin.

### Example 5

### Binding Assay

Recombinant HCV E2 protein or HIV-gp120-FITC, human, rabbit or bovine lipoproteins, HCV or mock virus preparations were added to cells for 60 min at 4° C. Cells were washed and incubated with ligand-specific antibody for 60 min at 4° C. Antibody binding was detected using goat anti-human IgG-Oregon green (10 μg/ml) for 45 min at 4° C. Cells were washed two times, fixed in PBS containing 4% paraformaldehyde, and analyzed by flow cytometry (FACScan, Becton Dickinson).

### Example 6

### HCV E2/Lipoprotein Interaction

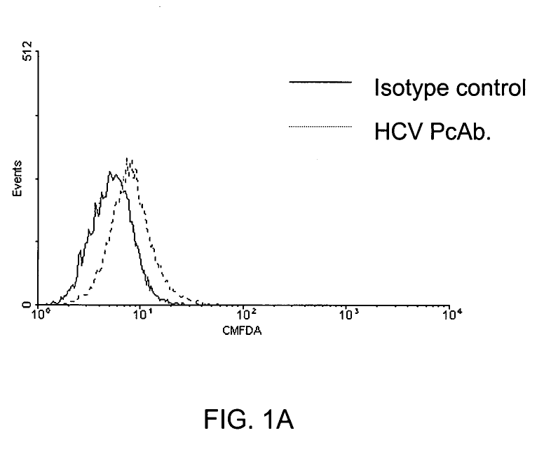

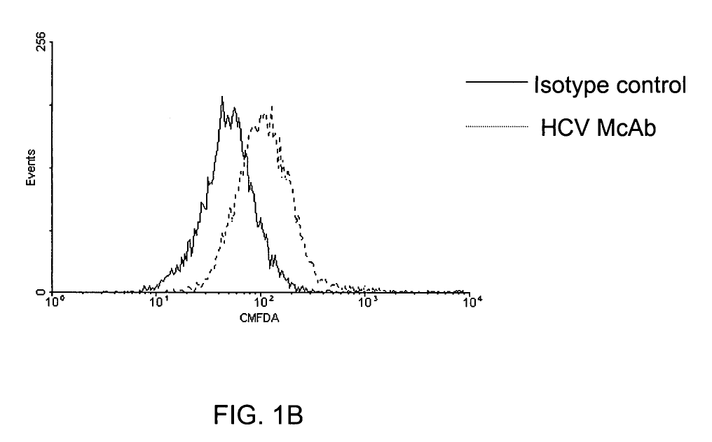

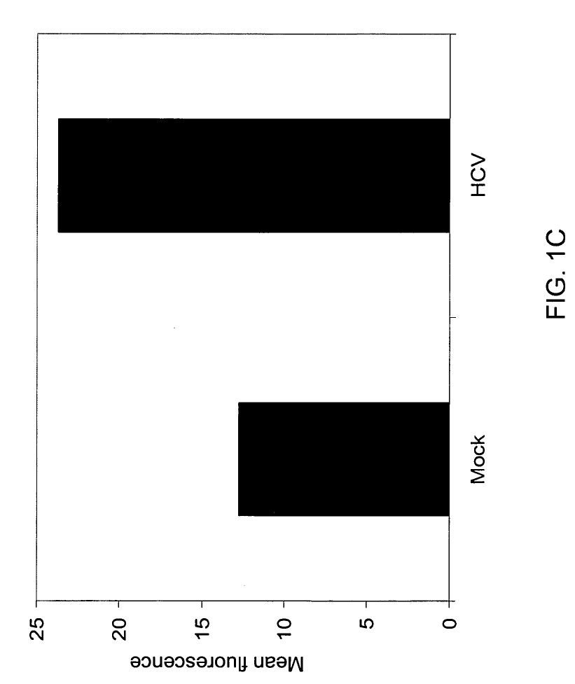

Plasma derived from HCV positive and HCV negative (Mock control) individuals with similar LDL cholesterol levels (104 and 105 g/dl respectively) were simultaneously separated on sucrose gradients and the same low density fractions were pelleted from each gradient. Mock and HCV preparations were incubated with MOLT 4 cells at 4° C., and cell-bound virus or LDL was detected using indirect immunofluorescence. Binding of HCV was detected in cells incubated with HCV preparations, but not when incubated with Mock control preparations (FIG. 1A and FIG. 1B). Cell-bound LDL was detected when both Mock- and HCV preparations were used. Higher levels of cell-bound LDL were repeatedly detected on cells incubated with HCV when compared with cells incubated with the control preparations (FIG. 1C).

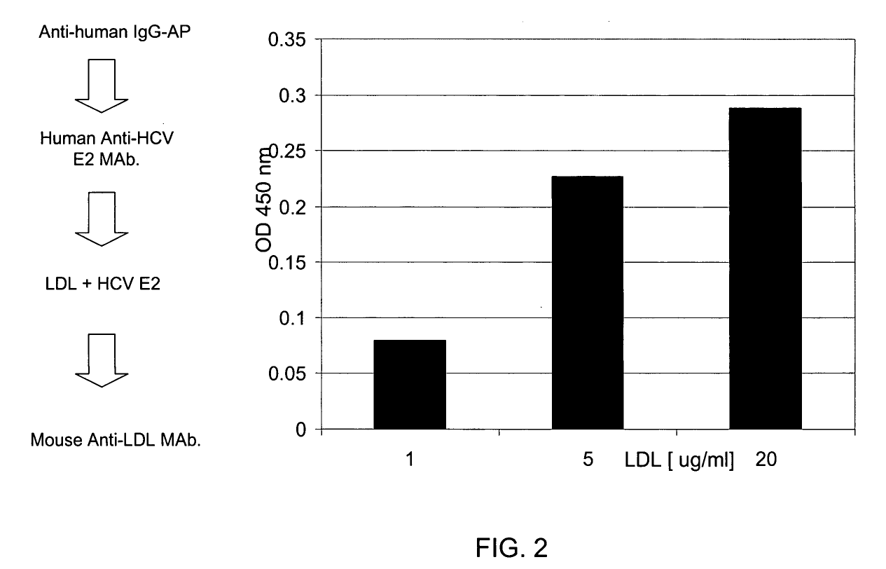

To determine if the increased levels of cell bound LDL identified in FIG. 1C were related to an association between HCV E2 and plasma lipoproteins, the inventors first utilized an ELISA method to assess E2-lipoprotein interactions. When murine McAb against human LDL was used as the capture antibody, HCV E2 was only detected when complexed with LDL. The extent of E2 captured was directly correlated to the LDL concentration used (FIG. 2).

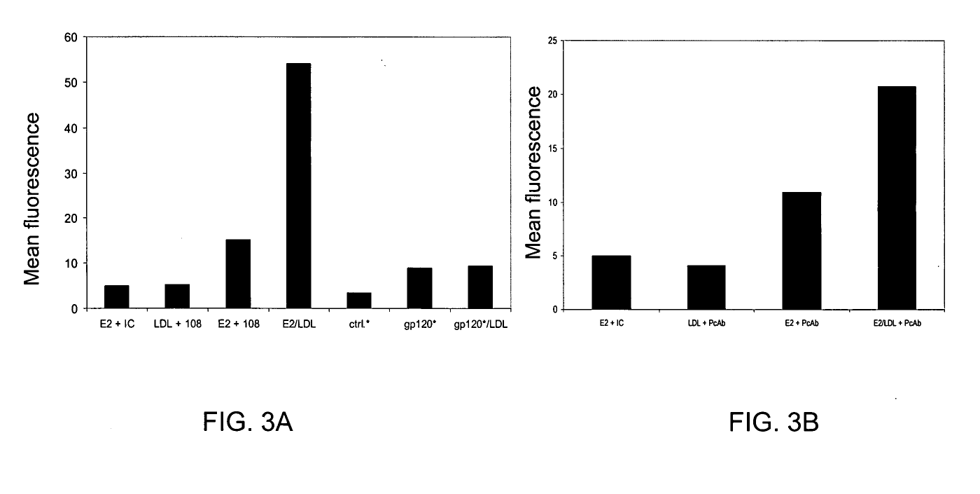

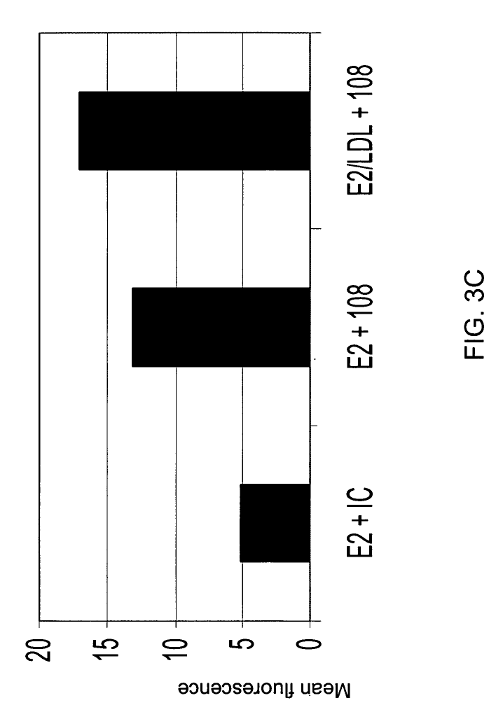

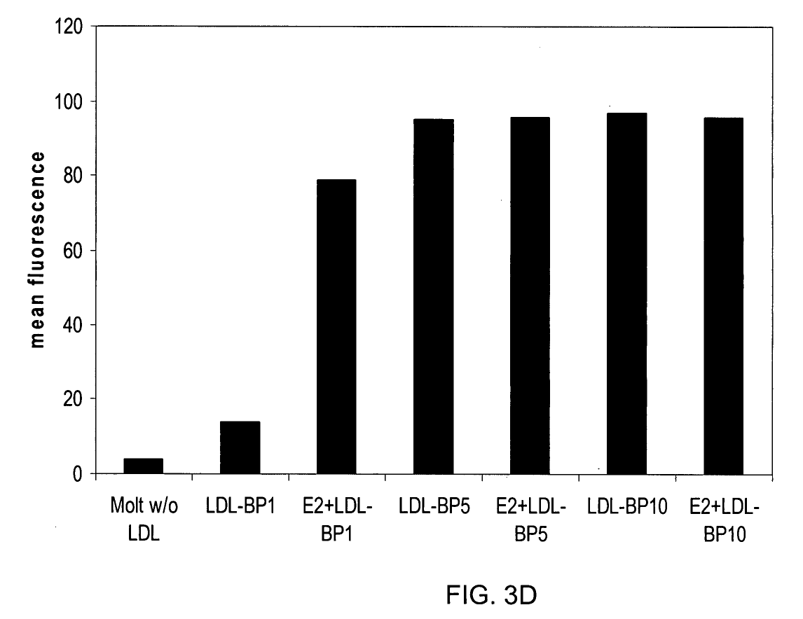

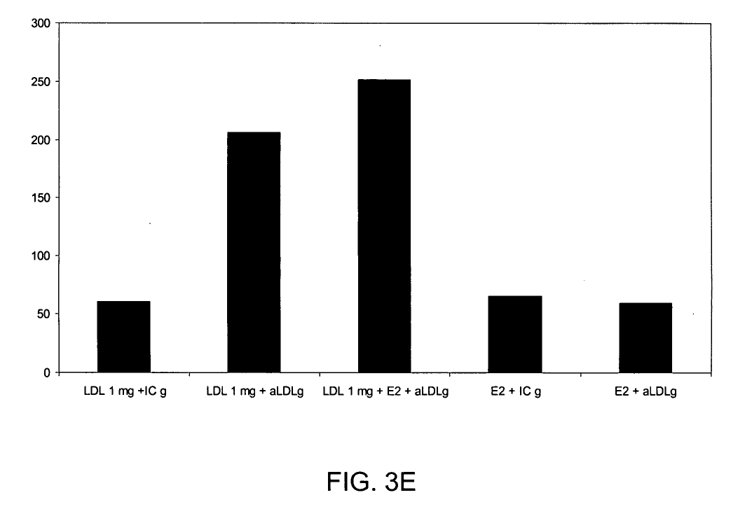

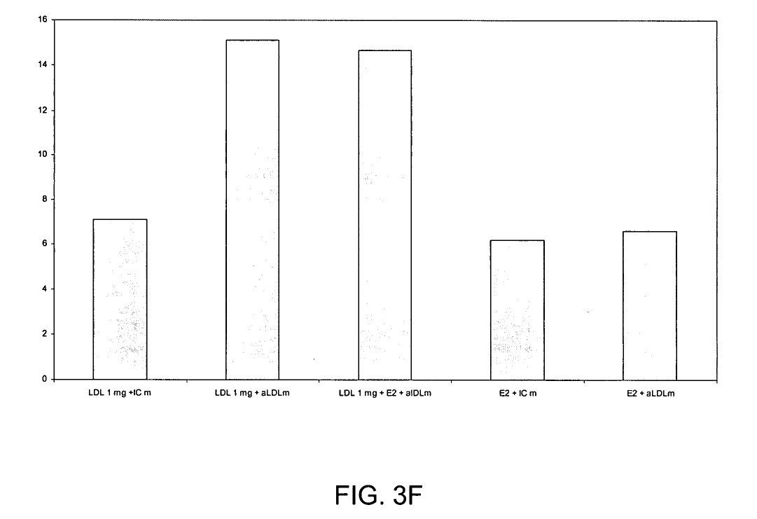

Because E2 interactions with human CD81 were shown to vary depending upon the context of CD81 presentation (Hadlock et al., 2000; Flint et al., 1999b; Wuenschmann 2000), the inventors evaluated the influence of LDL on E2 binding to MOLT-4 cells. HCV-E2 was incubated with increasing concentrations of human LDL for one hour at 4° C. prior to the addition of proteins to MOLT-4 cells. Following one hour at 4° C., cells were washed and HCV E2 binding was detected by flow cytometry. As previously shown, HCV-E2 bound to MOLT-4 cells (Wuenschmann et al., 2000), however the addition of LDL to HCV E2 increased the amount of E2 protein bound (FIG. 3A), and this effect was saturable at LDL concentrations of >2.5 μg/ml. The increase in E2 binding in the presence of LDL was seen when either the anti-HCV McAb or anti-HCV polyclonal anti-HCV were used for E2 detection (FIG. 3B). Thus this finding does not appear to be related to alterations in antibody affinity. To determine if HCV E2 influenced binding of human LDL to MOLT-4 cells, HCV-E2 and fluorescently labeled LDL were incubated for one hour at 4° C. and added to MOLT-4 cells for an additional hour. Cells were washed and analyzed for labeled-LDL binding (FIG. 3E and FIG. 3F). The amount of LDL bound reached saturation between 2.5 and 5 μg/ml (FIG. 3E and FIG. 3F), however, the amount of LDL bound increased when the LDL up to concentrations of 2.5 μg/ml was preincubated with HCV-E2 prior to addition to the cells (FIG. 3E and FIG. 3F).

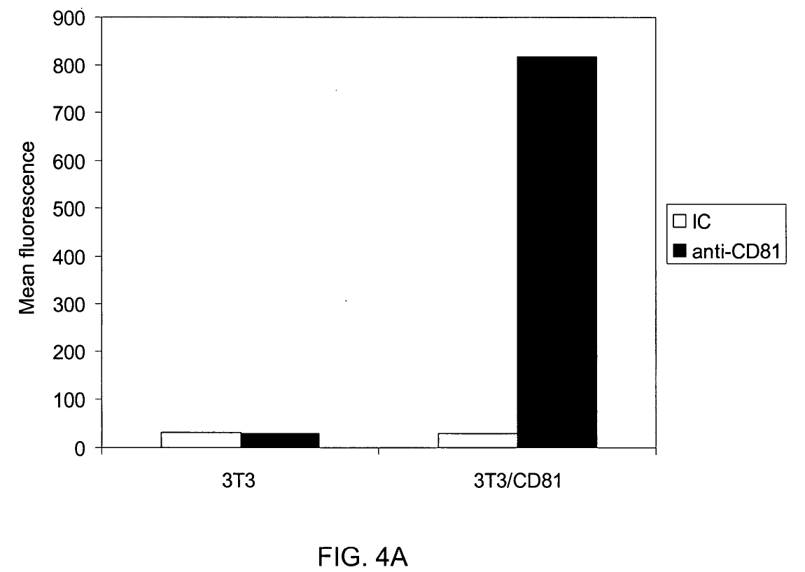

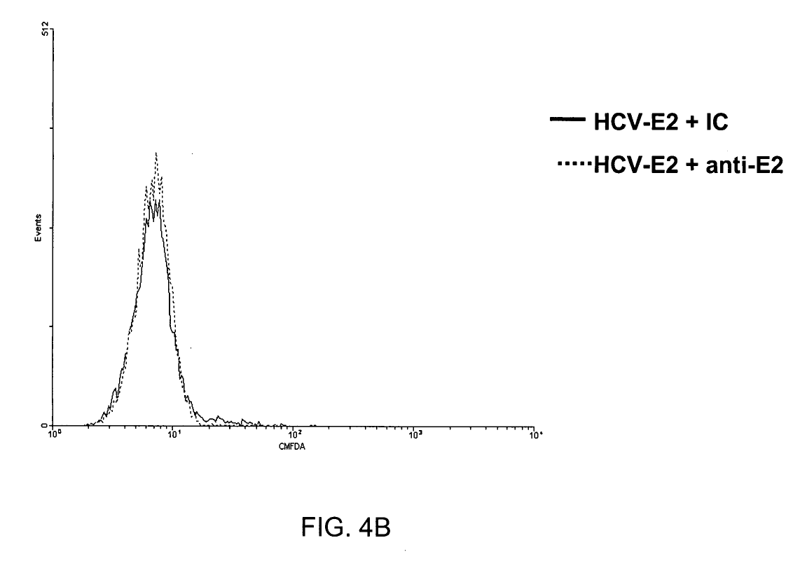

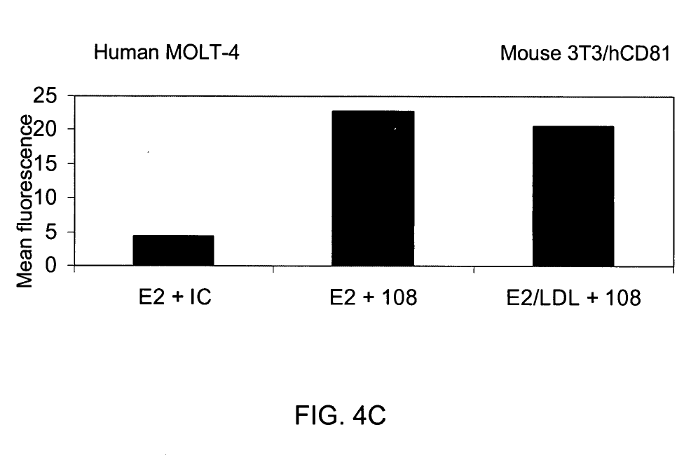

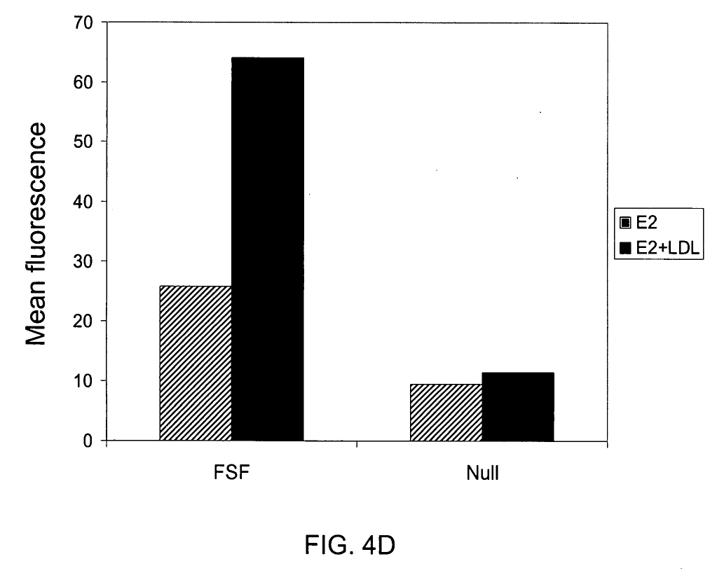

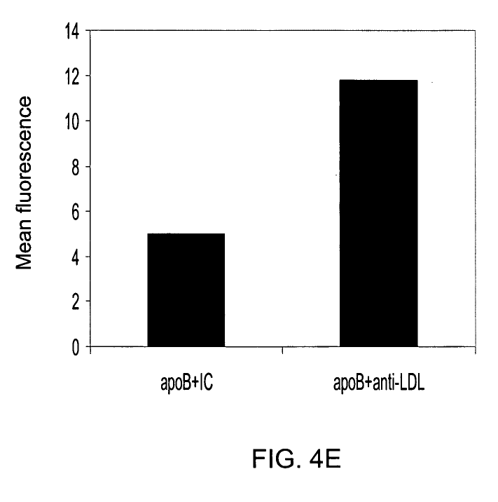

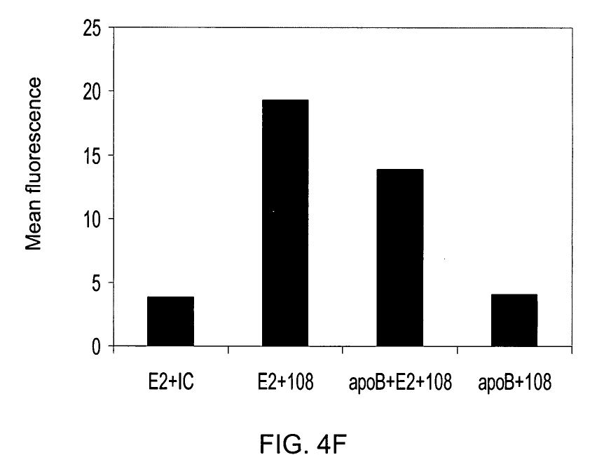

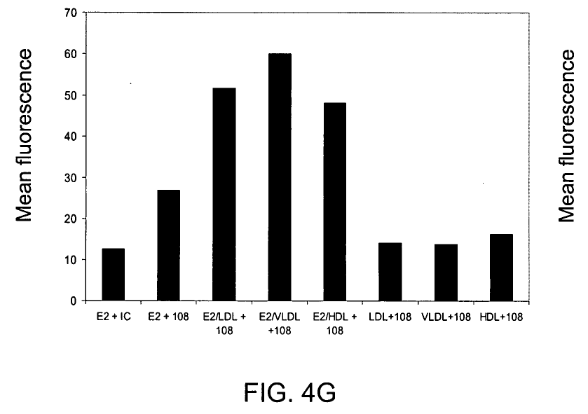

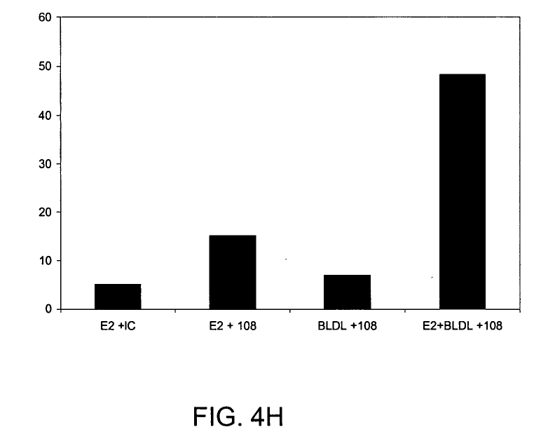

The increased level of binding of both proteins to MOLT-4 cells may be due to an association between LDL and HCV E2 that enables the resulting complex to bind to either the LDLr or to CD81. To evaluate this interaction further, mouse 3T3 cells expressing only murine CD81 or cells expressing both murine and human CD81 were studied. Mouse cells are not permissive for HCV, and murine CD81 does not interact with HCV E2 protein (Wuenschmann et al., 2000). FIG. 4A shows that 3T3 cells expressed high levels of human CD81 on the cell surface, whereas the control 3T3 cells did not. HCV E2 bound only to 3T3 cells expressing human CD81 (FIG. 4C), and preincubation of HCV-E2 with LDL did not increase the amount of cell bound HCV E2. Therefore, the enhanced E2 binding following incubation with LDL appears to require human LDLr expression on the cell surface. Similarly, HCV E2 bound to normal human foreskin fibroblasts (FSF) and to human fibroblasts that do not express the human LDLr (Null) (FIG. 4D). However, only cells expressing the LDLr demonstrated increased binding of HCV E2 when preincubated with LDL (FIG. 4D). Thus, the human CD81 molecule is necessary for HCV E2 binding to cells, but the increase in HCV-E2 binding after preincubation of E2 with LDL is dependent on the LDLr. It seems likely that the observed increase is due to an association between E2 and LDL, enabling the resulting complex to bind to the human LDLr in addition to human CD81.

To determine if HCV E2 interacts with the apoprotein or the lipid moiety of the human lipoprotein, the inventors evaluated apoprotein interactions with HCV E2. Binding of apoprotein B (apoB100) to the human LDLr was demonstrated using a polyclonal antibody against LDL (FIG. 4E). Preincubation of HCV E2 with apoprotein B did not increase the amount of cell-bound E2, suggesting that E2 does bind to the apoprotein of LDL (FIG. 4F). To determine if HCV-E2 interacts with lipoproteins other than LDL, HCV-E2 was incubated with human VLDL, LDL and HDL, as well as bovine lipoproteins. The amount of E2 bound to MOLT-4 cells increased with all these lipoproteins (FIG. 4G). This, and the lack of apoB interaction, suggests that HCV E2 interacts with the lipid moiety of the lipoproteins (FIG. 4H).

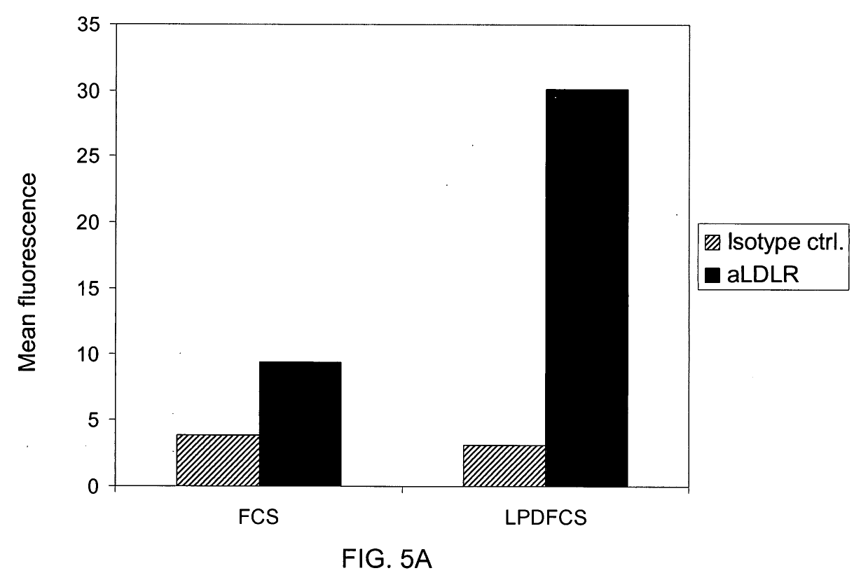

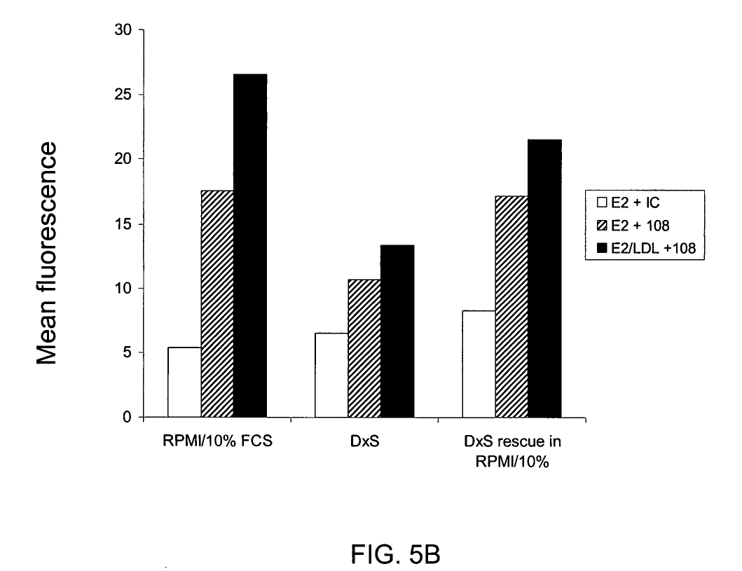

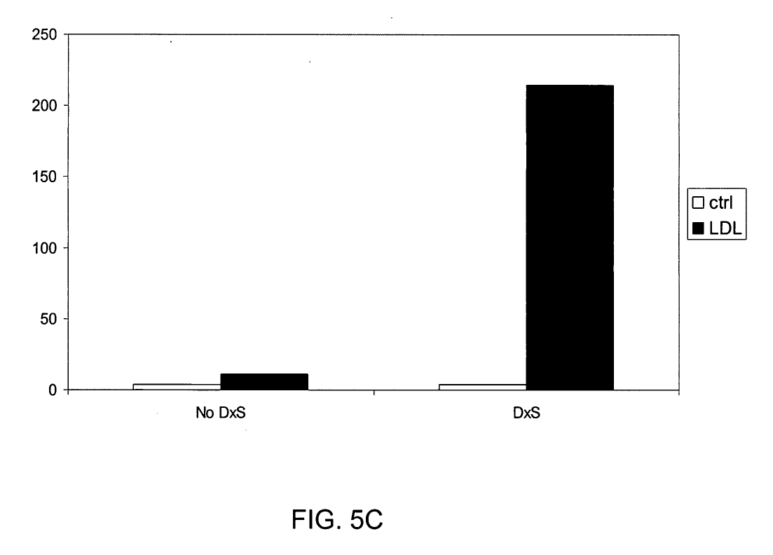

Cells used to study HCV binding are generally grown in the presence of lipoprotein-rich fetal calf serum, and these bovine serum lipoproteins may have an effect on HCV or HCV E2 binding. Depletion of bovine lipoproteins results in increased expression of the human LDLr (FIG. 5A) allowing binding of bovine LDL from tissue culture FCS (FIG. 5B), indicating that bovine FCS-derived lipoproteins bind to the human LDLr. To determine if the removal of bovine lipoproteins from the cell surface resulted in decreased HCV E2 binding, cell bound bovine lipoproteins were removed with dextran sulfate as described in Favre et al. (2001). FIG. 5C demonstrates that HCV E2 binding was decreased if MOLT-4 cells were stripped of lipoproteins by dextran sulfate, suggesting that HCV E2 binds to LDLr-bound bovine or human LDL in addition to binding to human CD81.

### Example 7

### HCV Mechanism for Attachment and Entrance into Target Cells

The mechanism by which HCV attaches and enters host cells has been poorly understood. HCV utilizes the low density lipoprotein receptor (LDLr) for cell binding and entry (Wuenschmann et al., 2000; Monazahian et al., 1999; Agello et al., 1999). The inventors have discovered that the HCV envelope glycoprotein (HCV E2 glycoprotein) binds to the lipid moiety of human lipoproteins, and the lipid-virus complex uses the natural receptor for LDL to bind to the cell. HCV enters the cell via endocytosis using the LDL receptor. HCV E2 glycoprotein interactions with LDL result not only in CD81-independent binding to cells (Wuenschmann et al., 2000), but also to enhancement in LDL binding and uptake by the cells.

Monazahian et al. (2000) demonstrated the formation of complexes between recombinant HCV envelope proteins E1/E2 and human, but not bovine LDL and HDL, whereas recombinant HCV core protein did not associate with human lipoproteins. In this study, the inventors demonstrated an interaction between HCV E2 (aa 384-715) (SEQ. ID. NO: 3) and LDL both in ELISA and cell binding studies.

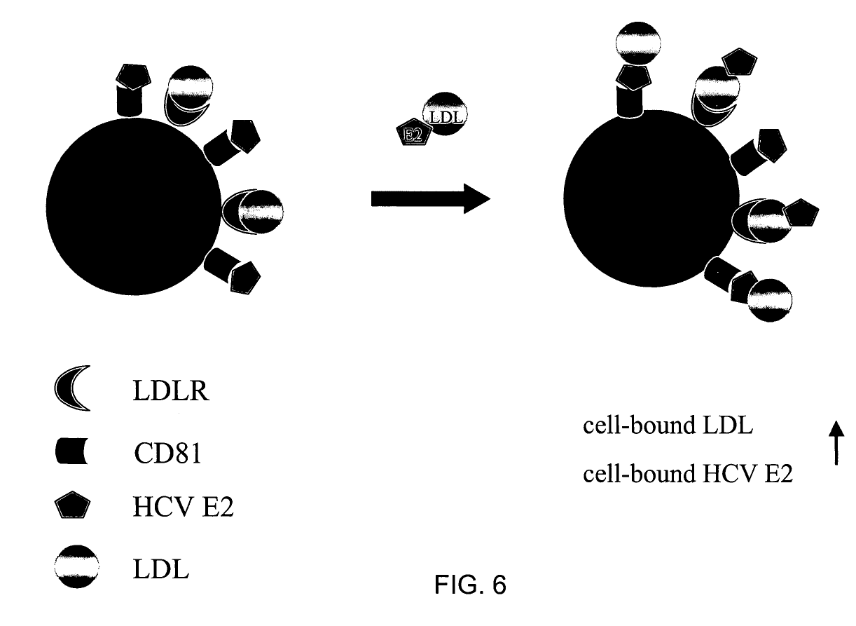

HCV E2 also binds specifically to human CD81, which is widely expressed on human cells. LDL and VLDL bind to the human LDLr, which is also widely expressed. The present data indicate that the incubation of HCV E2 with LDL increased binding of both molecules to MOLT-4 cells, suggesting that the E2-LDL complex may bind to either the LDLr or to human CD81 (FIG. 6). Both receptors are involved in complex binding, as experiments using mouse 3T3 cells, expressing only human CD81 and human Null cells, expressing human CD81, but not LDLr bound E2-LDL complexes equally to E2 alone.

The interaction between HCV E2 and LDL did not appear to be based on protein-protein interactions, since HCV E2 binding was not increased following incubation with apoprotein apoB100, even though apoB100 binding to the LDLr was demonstrated. This was further supported by experiments showing that the amount of HCV E2 bound to MOLT-4 cells was increased by pre-incubation with human lipoproteins VLDL, LDL and HDL, all of which contain different apoproteins. Apoprotein apoB100 is present in VLDL and LDL, whereas apoprotein E is present in VLDL and HDL. Both ApoB100 and apoE are ligands of the human LDLr. Addition of bovine lipoproteins also increased binding of HCV E2, suggesting that the lipid-moiety of these lipoproteins is interacting with HCV E2. The data confirm previous studies suggesting that HCV E2 interacts with all human serum lipoproteins. HCV particle types of low, intermediate and high density have been described (Xiang et al., 1998; Bradley et al., 1991; Hijikata et al., 1993; Bradley et al., 1983). Whereas the low density particles were found to be infectious and are presumably associated with VLDL and LDL, it has been suggested that denser HCV particles represent nucleocapsids or virus-IgG complexes (Xiang et al., 1998; Han et al., 1997). The finding of an association between E2 and HDL suggests that the intermediate density HCV particles may be comprised of the HCV-HDL complexes, since the density of these particles approximate that of HDL (1.11-1.17 g/cm³). These intermediate particles do not appear to be infectious in humans (Bradley, 2000; Bradley et al., 1985; Shimizu et al., 1993), and they did not bind to permissive cells in vitro (Agnello et al., 1999; Wuenschmann et al., 2000).

The interaction of HCV E2 with bovine lipoproteins complicates interpretation of virus binding assays, since bovine lipoproteins from fetal calf serum influenced HCV E2 binding to cells. FCS-derived bovine lipoproteins bound to the human LDLr and were able to bind HCV E2 (FIG. 4H). Treatment of cells with dextran sulfate removed all cell-bound LDL originating from the cell culture medium and resulted in decreased E2 binding. This may explain previous reports demonstrating HCV E2 binding to CD81 negative cells as these cells were grown in FCS (Hamaia et al., 2001; Flint et al., 1999). Favre et al. (2001) recently demonstrated that removal of cell-bound LDL, prior to infection with HCV containing serum, increased the efficiency of infection significantly, further suggesting that bovine lipoproteins bound to the human LDLr blocked HCV attachment and entry.

The interaction of HCV E2 with human lipoproteins further supports the role of the LDLr as receptor for HCV, but nevertheless CD81 does bind HCV E2 as well as E2-LDL complexes. Therefore CD81 and the LDLr receptor may be required to act in concert for binding and entry of HCV.

Although CD81 and the LDLr are widely distributed in human cells, and thus do not provide a cell-specific receptor for HCV, the liver is the site of more than 40% of LDL uptake, thus HCV would preferentially be taken out of the circulation in the liver if LDLr is used for virus attachment and entry. It is likely that additional factors influence tissue tropism, as many viruses demonstrate specific tissue tropism in spite of having a cell receptor that is present on many cell types (Scheider-Schaulies, 2000; Lusso, 2000; Evans and Almond, 1998).

### Example 8

### Correlation Between HCV Infection and LDL Levels

Data generated in the VA laboratory (Wuenschmann et al., 2000; Wuenschmann and Stapleton, 2002), and epidemiological data generated in the Iowa City VA and in collaboration with the VA CSG3 study (Polgreen et al., 2002; Stapleton et al., 2002) strongly suggest that infection with HCV alters lipid metabolism, and that the use of the “statin” class of drugs will enhance HCV cell attachment and entry (Wuenschmann et al., 2000). While HIV protease inhibitors (PI) induce an increase in serum lipoproteins that is associated with endothelial dysfunction (Stein et al., 2001), the effects of lipid-lowering interventions on endothelial dysfunction and on flavivirus co-infection have not been evaluated. Also, several protease inhibitors are to be taken with high fat diets to improve absorption. The effect of a high lipid diet on HCV or GBV-C replication is unknown.

The prognosis for HIV-infected people living in developed countries has improved remarkably over the past 6 years, in large part due to the development and widespread use of potent combinations of antiretroviral therapy. While treatment has greatly improved survival, regimens containing HIV PIs have been associated with the development of several risk factors for coronary artery disease, including hyperlipidemia, hyperglycemia and endothelial dysfunction (Stein et al., 2001). Due to the improved efficacy of antiretroviral therapy, liver disease and cardiovascular disease related to therapy are increasingly common causes of mortality among HIV-positive individuals (Bica et al., 2001; Wolfe et al., 2002). Due to shared modes of transmission, HIV infected people are frequently co-infected with one or more human Flaviviruses. Up to 90% of HIV-positive people who acquire HIV through intravenous drug use are co-infected with HCV (Thomas et al., 1996), and up to 40% of HIV infected individuals are co-infected with another persistent human Flavivirus GBV-C (10). GBV-C—HIV co-infected patients appear to have prolonged survival compared with HIV patients who are not actively infected with GBV-C (Xiang et al., 2001).

Stein et al. (2001) demonstrated that HIV PI use increased total cholesterol, low density lipoprotein (LDL) cholesterol, triglycerides, and impaired flow-mediated vasodilation (FMD) indicating endothelial dysfunction. The effects of lipid-lowering interventions in this population on either lipid abnormalities or on endothelial dysfunction were not evaluated. The inventors and 2 other groups showed that HCV utilizes the LDL receptor (LDLr) for cell binding and entry (Wuenschmann et al., 2000; Monazahian et al., 1999; Agello et al., 1999). Recent work in the laboratory identified a novel mechanism for HCV attachment and entry, demonstrating that the HCV envelope glycoprotein (E2) binds to the lipid moiety of human lipoproteins, and the lipid-virus complex uses the natural receptor for LDL to bind to the cell (Wuenschmann and Stapleton, 2002). HCV enters the cell via endocytosis using the LDL receptor. E2 interactions with LDL result not only in CD81-independent binding to cells, but also to enhancement in LDL binding and uptake by the cells (Wuenschmann et al., 2000). Since the inventors demonstrated that HCV binding to cells was directly related to the extent of LDLr expression on the cell surface, and the commonly used lipid-lowering drugs in the “statin” class work by up-regulating LDLr expression, these drugs will enhance HCV binding and uptake to hepatocytes. Currently, certain statins that interact minimally with PIs are the recommended first-line treatment for hypercholesterolemia in HIV-positive people (www.nhlbi.nig.gov.guidelines/cholesterol/index.htm). Fish oil (or n-3 polyunsaturated fatty acids) lower LDL and cholesterol levels by interrupting intracellular VLDL synthesis, the precursor molecule for LDL (Wang et al., 1993). Thus the mechanism of action of fish oil therapy for hyperlipidemia is different from that of the statins, and would not be expected to enhance virus binding and entry. Few data exist regarding GBV-C—cell interactions, although one study provided some data to suggest that GBV-C also uses the LDLr to enter cells (Agello et al., 1999). If true, alterations in LDLr expression will also influence GBV-C replication, which may prove to be beneficial for HIV-GBV-C co-infected individuals.

The inventors recently carried out two epidemiological studies to test the hypothesis that HCV infection influences LDL levels in humans. The inventors studied two midwestern HIV clinic populations and found that there was significantly less risk of HIV-HCV co-infected individuals developing hypercholesterolemia when compared to those infected only with HIV (p=0.04, n=817; Stapleton et al., 2002). Based on these results, the inventors collaborated with Dr. Amy Justice and the Veterans Aging cohort study 3 to determine the LDL and cholesterol levels (Polgreen et al., 20024). Multivariate linear regression revealed that HCV infection was independently associated with lower LDL cholesterol (p<0.001; n=409) and total cholesterol levels (p<0.001; n=606), whereas HIV PI use was associated with higher LDL and total cholesterol (p<0.001 for both). Neither HCV infection nor PI use was significantly associated with HDL cholesterol level (n=408), and there was no association between cholesterol levels and the use of lipid-lowering medications, age, gender, HIV transmission category, HIV RNA levels, ALT or AST levels. These data confirmed that HCV infection is significantly associated with lower serum LDL and total cholesterol levels, and support older literature showing that patients with chronic hepatitis have lower rates of coronary artery disease (Hall et al., 1953; Creed et al., 1955).

The finding of reduced atherosclerosis and lower lipid levels has been ascribed to decreased VLDL synthesis secondary to liver disease (Plotkin et al., 2000), although this has not been carefully studied in patients with subclinical HCV infection. In the only study published to date addressing the specificity of chronic active viral hepatitis and low cholesterol levels, HCV was shown to be associated with a significant decrease in cholesterol levels when compared with patients with chronic active hepatitis B virus infection (Fabris et al., 1997). To determine if low LDL and cholesterol levels were related to the extent of HCV-related liver disease, the inventors studied subjects from the ICVA Liver Clinic for whom liver biopsy data and serum lipid studies were available. Using the Knodell grading score for fibrosis (0=absent, 1=minimal, 2=mild, 3=moderate and 4=severe, neither total cholesterol (n=89) nor LDL cholesterol (n=64) were associated with the extent of liver disease, nor were there any differences in mean lipid levels related to stage of fibrosis (Polgreen, et al., 2002). A case-control study of lipid levels is underway to better characterize this finding.

### Example 9

### Future Studies

The inventors conclude from the above information that atherosclerosis and lipid abnormalities in HIV-infected patients are an increasing cause of morbidity and mortality, and that liver disease in HIV-HCV co-infected people may be influenced by the use of the currently recommended treatment of choice for hyperlipidemia (www.nhlbi.nig.gov.guidelines/cholesterol/index.htm). Thus, this proposal is designed to:

(a) Compare two regimens for treatment of hyperlipidemia in HIV-positive people. The secondary scientific objectives will also allow investigation into the relationship between hyperlipidemia and flavivirus replication and pathogenesis. To accomplish these objectives, the inventors propose a prospective, randomized, cross-over clinical trial.

(b) Enrollment will include HIV-positive subjects recruited from the Iowa City VAMC and the University of Iowa HIV/AIDS clinics on stable HIV therapy regimens, the Iowa City VA Liver Clinic for the HIV-negative HCV-positive control group, and from the IC VAMC General Internal Medicine Clinics for the matched, HIV-negative, HCV-negative control group.

(c) Four groups of patients will be studied. HIV-positive subjects will be stratified by their treatment regimen into those taking protease inhibitors (PI+) or those not using PIs (no PI). These subjects will be compared with control groups of age-matched HIV negative subjects with and without HCV infection.

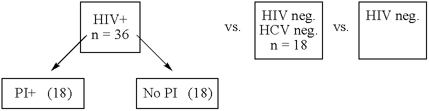

Base Populations: The principal investigator for this application is one of three staff physicians for the ICVAMC HIV clinic, which provides primary care for 55HIV+ patients. In addition, he is director of the UI HIV/AIDS clinic (362 patients cared for in 2001). There is a strong track record of enrollment in HIV-related clinical trials from these two clinics. The Medical Director of the VAMC Liver Clinic, Dr. Warren Schmidt, will be a co-investigator and has provided care to more than 250 veterans with HCV-related liver disease during the past 18 months. The Director of General Internal Medicine (Dr. Gary Rosenthal) at the ICVAMC has agreed to facilitate the recruitment of the HIV-negative, HCV-negative control group.

Interventions: 1. Baseline measurements will be obtained twice prior to interventions (day minus 7-14 and day 0). 2. All will be randomly assigned to receive atorvastatin (10 mg daily) (Group A) or fish oil (3.6 g/day) (Group B) (week 0). Subjects will be monitored biweekly for both toxicity and study measurements. Subjects will stop their atorvastatin or fish oil on week 4. 3. 4 weeks after stopping their lipid lowering agent (week 8), Group A subjects will receive fish oil, and Group B subjects will receive atorvastatin for 4 additional weeks (week 12). 4. Four weeks after the second treatment period (week 16), end-of study measurements will be performed. 5. Selected HCV and GBV-C positive subjects who do not have hyperlipidemia meeting levels prompting treatment under current guidelines will be provided a high fat diet for 14 days and repeat measurements of endothelial dysfunction and viral replication will be obtained on weekly.

(d) Endpoints evaluated. The primary endpoint will be to determine the intervention (atorvastatin vs. fish oil) that most effectively reduces cholesterol levels and improves endothelial dysfunction. This will include both reduction in lipid levels and evidence that HCV replication is not enhanced. Measurements will include the determination of baseline fasting lipids (cholesterol, HDL, triglycerides), glucose levels, insulin levels, liver-associated enzymes (ALT,AST,Bili,alkaline phosphatase, GGT). The clinical tests will be performed by the Iowa City VA clinical laboratory. Brachial artery resistance measurements will be performed in the lab of Dr. William Haynes, director of the University of Iowa GCRC. Dr. Haynes has extensive experience with these assays (Christoffersen and Junge, 1991). HCV and GBV-C detection and quantification will be determined by using real-time RT-PCR measurements as previously described (Wuenschmann et al., 2000; Xiang et al., 2001).

(e) Data Collection Summary: Tolerability and safety will be evaluated on each visit. Two baseline (pre-intervention) laboratory analyses will be performed. Safety monitoring, lipid levels, and plasma for viral quantification will be monitored weekly. Brachial artery resistance measurements will be carried out on week 4, 8, 12, and 16.

(f) Analytic Plan: Methodology is highly controlled to maintain minimal analytical variability, and the VAMC Clinical laboratory is CAP accredited. HCV and GBV-C RNA detection and quantitation by RT-PCR (and real-time PCR™) is highly reproducible within a single experiment. To ensure that inter-experiment variation does not occur, all RNA comparisons will be done in triplicate in a batch analysis on samples stored appropriately. Several standard statistical methods will be used. For example, the inventors make extensive use of the F test for sample variance, followed by the t-test for equal or unequal variances as appropriate. These methods require an assumption about the normality of sample distributions, so the inventors will also use non-parametric (Mann-Whitney U) tests (which requires no such assumption when necessary). Several comparisons with different statistical power can be obtained, and sample size estimates have been evaluated. Assume for example, 12 subjects before and after atorvastatin, but these are the same 12 subjects at different times. Thus, a t-test for paired sample means must be used, yielding 11 degrees of freedom (dF). In this comparison, t=√n (mean difference)/standard deviation. Estimates from this relationship yield crude values of the required (tcrit_{0.05}=1.796 single-tailed) differences between the sample means before and after atorvastatin of somewhat over 0.5 standard deviations. A more rigorous level (tcrit_{0.01}=2.718) requires mean difference closer to 1.0 S.D. A more general analysis with similar assumptions of differences in the measured parameters (for example, LDL or log10 HCV RNA) for two independent arms, with pooled S.D. of 0.75 and mean difference between arms of 1 S.D. requires, with 90% power n=12 at α=0.025; n=15 at α=0.01; and n=17 at α=0.005. The inventors will thus need to recruit 18 subjects for each arm to allow for some drop-out, although based on previous experience with more than 250 HIV-infected subjects in >20 clinical trials, the inventors expect minimal drop out. Individual subject data will all be plotted with respect to time, disease and intervention status, and treatment, to further display any patterns of variation. Statistical design and methods were reviewed by Dr. Leon Burmeister, Professor and Head of the statistical section of the University of Iowa Clinical Research Center. If these studies are funded, the Iowa CRC will provide ongoing statistical support.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 4,196,265

U.S. Pat. No. 4,282,287

U.S. Pat. No. 4,542,102

U.S. Pat. No. 4,659,774

U.S. Pat. No. 4,682,195

U.S. Pat. No. 4,683,202

U.S. Pat. No. 4,683,202

U.S. Pat. No. 4,797,368

U.S. Pat. No. 4,816,571

U.S. Pat. No. 4,959,463

U.S. Pat. No. 5,139,941

U.S. Pat. No. 5,141,813

U.S. Pat. No. 5,252,743

U.S. Pat. No. 5,264,566

U.S. Pat. No. 5,384,253

U.S. Pat. No. 5,412,087

U.S. Pat. No. 5,428,148

U.S. Pat. No. 5,445,934

U.S. Pat. No. 5,554,744

U.S. Pat. No. 5,574,146

U.S. Pat. No. 5,580,859

U.S. Pat. No. 5,589,466

U.S. Pat. No. 5,602,244

U.S. Pat. No. 5,633,388

U.S. Pat. No. 5,645,897

U.S. Pat. No. 5,656,610

U.S. Pat. No. 5,702,932

U.S. Pat. No. 5,705,629

U.S. Pat. No. 5,736,524

U.S. Pat. No. 5,780,448

U.S. Pat. No. 5,789,215

U.S. Pat. No. 5,902,831

U.S. Pat. No. 5,925,565

U.S. Pat. No. 5,928,906

U.S. Pat. No. 5,935,819

U.S. Pat. No. 5,945,100

U.S. Pat. No. 5,981,274

U.S. Pat. No. 5,994,136

U.S. Pat. No. 5,994,624

U.S. Pat. No. 6,013,516

U.S. Pat. No. 6,326,151

Adult Treatment Panel III. 2001. Executive summary of the third report of the expert panel on detection, evaluation, and treatment of high blood cholesterol in adults. (www.nhlbi.nig.gov.guidelines/cholesterol/index.htm).

Agello et al., PNAS, 96:12766-12771, 1999.

Almendro et al., J Immunol., 157:5411-5421, 1996.

Alter, Hepatology, 26:62S-65S, 1997.

Angel et al., Mol. Cell. Biol., 7:2256, 1987.

Angel et al., Cell, 49:729, 1987b.

Angel et al., Mol. Cell. Biol., 7:2256, 1987a.

Atchison and Perry, Cell, 46:253, 1986.

Atchison and Perry, Cell, 48:121, 1987.

Ausubel et al., In: Current Protocols in Molecular Biology, John, Wiley & Sons, Inc, New York, 1994.

Baichwal and Sugden, In: Gene Transfer, Kucherlapati (ed.), New York, Plenum Press, 117-148, 1986.

Banerji et al., Cell, 27(2 Pt 1):299-308, 1981.

Banerji et al., Cell, 33(3):729-740, 1983.

Barany and Merrifield, In: The Peptides, Gross and Meienhofer (eds.), Academic Press, New York, 1-284, 1979.

Bartenschlager and Lohmann, J. Gen. Virol, 81:1631-1648, 2000.

Bennett et al., Journal of Molecular Recognition, 8: 52-58 (1995).

Berkhout et al., Cell, 59:273-282, 1989.

Bica et al., Clin. Infect. Dis., 32:492-497, 2001.

Blanar et al., EMBO J., 8:1139, 1989.

Blomer et al., J. Virol., 71(9):6641-6649, 1997.

Bodine and Ley, EMBO J., 6:2997, 1987.

Boshart et al., Cell, 41:521, 1985.

Bosze et al., EMBO J., 5(7):1615-1623, 1986.

Braddock et al., Cell, 58:269, 1989.

Bradley, Curr. Top. Microbiol. Immunol., 242:1-23, 2000.

Bradley et al., J. Infect. Dis., 148:6547-6549, 1983.

Bradley et al., Gastroenterology, 88:773-779. 1985.

Bradley et al., J. Med. Virol., 34:206-208, 1991.

Bulla and Siddiqui, J. Virol., 62:1437, 1986.

Burden and Von Knippenberg (Eds.), Elseview, Amsterdam, pp. 71-74; 75-83, 1984.

Campbell and Villarreal, Mol. Cell. Biol., 8:1993, 1988.

Campbell, Monoclonal Antibody Technology, Laboratory Techniques in Biochemistry and Molecular Biology, Vol. 13, 1984.

Campere and Tilghman, Genes and Dev., 3:537, 1989.

Campo et al., Nature, 303:77, 1983.

Carbonelli et al., FEMS Microbiol Lett, 177(1):75-82, 1999.

Celander and Haseltine, J. Virology, 61:269, 1987.

Celander et al., J. Virology, 62:1314, 1988.

Chandler et al., Cell, 33:489, 1983.

Chandler et al., Proc Natl Acad Sci USA, 94(8):3596-601, 1997.

Chang et al., Mol. Cell. Biol., 9:2153, 1989.

Chatterjee et al., Proc Natl. Acad. Sci. U.S.A., 86:9114, 1989.

Chen and Okayama, Mol. Cell Biol., 7(8):2745-2752, 1987.

Choi et al., Cell, 53:519, 1988.

Christoffersen and Junge, APMIS, 23:40-45, 1991.

Cocea, Biotechniques, 23(5):814-816, 1997.

Cohen et al., J. Cell. Physiol., 5:75, 1987.

Coligan et al., Current Protocols in Immunology, 1(2): Ch. 5 (1991).

Conry-Cantilena et al., N. Engl. J. Med., 334:1691-1695, 1996.

Costa et al., Mol. Cell. Biol., 8:81, 1988.

Cotton et al., Proc Natl Acad Sci USA, 89(13):6094-6098, 1992.

Coupar et al., Gene, 68:1-10, 1988.

Creed et al., Am. J. Med. Sci., 230:385-389, 1955.

Cripe et al., EMBO J., 6:3745, 1987.

Culotta and Hamer, Mol. Cell. Biol., 9:1376, 1989.

Curiel, Nat Immun, 13(2-3):141-64, 1994.

Dandolo et al., J. Virology, 47:55-64, 1983.

De Villiers et al., Nature, 312(5991):242-246, 1984.

Department of Veterans Affairs—Press Release. New VA research looks at alcohol and HIV interaction. 11-27; 2001.

Deschamps et al., Science, 230:1174-1177, 1985.

Dunlap, Immobilized Biochemicals and Affinity Chromatography, Adv. Exp. Med. Biol. 42 ed., Plenum Press, N.F. 1974.

Edbrooke et al., Mol. Cell. Biol., 9:1908, 1989.

Edlund et al., Science, 230:912-916, 1985.

European Patent App. 266,032

Evans and Almond, Trends Microbiology, 6:198-202, 1998.

Fabris et al., Clinic. Chimica. Acta, 261:159-165, 1997.

Favre et al., Sciences de la Vie. 324(12):1141-8, December 2001.

Fechheimer et al., Proc. Nat'l Acad. Sci. USA, 84:8463-8467, 1987.

Feng and Holland, Nature, 334:6178, 1988.

Firak and Subramanian, Mol. Cell. Biol., 6:3667, 1986.

Flint and McKeating, Rev. Med. Virol., 10: 101-117, 2000.

Flint et al., J. Virol., 73:6235-6244, 1999b.

Flint et al., J. Virol, 73:6782-6790, 1999a.

Foecking and Hofstetter, Gene, 45(1): 101-105, 1986.

Fraley et al., Proc. Nat'l Acad. Sci. USA, 76:3348-3352, 1979.

Friedmann, Science, 244:1275-1281, 1989.

Fujita et al., Cell, 49:357, 1987.

Gilles et al., Cell, 33:717, 1983.

Gloss et al., EMBO J., 6:3735, 1987.

Godbout et al., Mol. Cell. Biol., 8:1169, 1988.

Goding, In: Monoclonal Antibodies: Principles and Practice, 2d ed., Academic Press, Orlando, Fla., pp. 60-61, and 71-74, 1986.

Goodboum and Maniatis, Proc. Nat'l Acad. Sci. USA, 85:1447, 1988.

Goodboum et al., Cell, 45:601, 1986.

Graham and van der Eb, Virology, 52:456-467, 1973.

Greene et al., Immunology Today, 10:272, 1989

Grosschedl and Baltimore, Cell, 41:885, 1985.

Grunhaus et al., Seminar in Virology, 200(2):535-546, 1992.

Hadlock et al., J. Virol., 74:10407-10416, 2000.

Hall et al., Am. J. Pathol., 9:993-1023, 1953.

Hamaia et al., Blood, 98:2293-2300, 2001.

Han et al., In Viral Hepatitis and Liver Disease, Rizzeto et al., (Eds.),. Edizioni Minerva, Medica, Turin. 228-231, 1997.

Harland and Weintraub, J. Cell Biol., 101:1094-1099, 1985.

Harlow and Lane, Antibodies: A Laboratory manual, Cold Spring Harbor Laboratory, 1988.

Haslinger and Karin, Proc. Nat'l Acad. Sci. USA, 82:8572, 1985.

Hauber and Cullen, J. Virology, 62:673, 1988.

Hen et al., Nature, 321:249, 1986.

Hensel et al., Lymphokine Res., 8:347, 1989.

Herr and Clarke, Cell, 45:461, 1986.

Hijikata et al., J. Virol., 67:1953-1958, 1993.

Hirochika et al., J. Virol., 61:2599, 1987.

Hirsch et al., Mol. Cell. Biol., 10:1959, 1990.

Hobbs et al., N. Engl. J. Med., 317:734-737, 1987.

Holbrook et al., Virology, 157:211, 1987.

Horlick and Benfield, Mol. Cell. Biol., 9:2396, 1989.

Horwich et al., Virol., 64:642-650, 1990.

Houghten et al., Proc. Nat'l Acad. Sci. USA 82:5131-5135, 1985.

Huang et al., Cell, 27:245, 1981.

Hug et al., Mol. Cell. Biol., 8:3065, 1988.

Hug et al., Mol Cell Biol, 8(8):3065-79, 1988.

Hwang et al., Mol. Cell. Biol., 10:585, 1990.

Imagawa et al., Cell, 51:251, 1987.

Imbra and Karin, Nature, 323:555, 1986.

Imler et al., Mol. Cell. Biol, 7:2558, 1987.

Imperiale and Nevins, Mol. Cell. Biol., 4:875, 1984.

Inouye and Inouye, Nucleic Acids Res., 13: 3101-3109, 1985.

Jakobovits et al., Mol. Cell. Biol., 8:2555, 1988.

Jakoby and Wilchek, Meth. Enz., Part B, 34 ed., Acad. Press, N.Y. 1974.

Jameel and Siddiqui, Mol. Cell. Biol., 6:710, 1986.

Jaynes et al., Mol. Cell. Biol., 8:62, 1988.

Johanson et al., The Journal of Biological Chemistry, 270, (16): 9459-9471 (1995).

Johnson et al., Mol. Cell. Biol., 9:3393, 1989.

Kadesch and Berg, Mol. Cell. Biol., 6:2593, 1986.

Kaneda et al., Science, 243:375-378, 1989.

Karin et al., Mol. Cell. Biol., 7:606, 1987.

Karin et al., Mol. Cell. Biol., 7:606, 1987.

Katinka et al., Cell, 20:393, 1980.

Kato et al., J. Biol. Chem., 266:3361-3364, 1991.

Kawamoto et al., Mol. Cell. Biol., 8:267, 1988.

Kelleher and Vos, Biotechniques, 17(6):1110-7, 1994.

Kiledjian et al., Mol. Cell. Biol., 8:145, 1988.

Klamut et al., Mol. Cell. Biol., 10:193, 1990.

Koch et al., Mol. Cell. Biol., 9:303, 1989.

Kraus et al., FEBS Lett, 428(3):165-70, 1998.

Kriegler and Botchan, In: Eukaryotic Viral Vectors, Gluzman, ed., Cold Spring Harbor: Cold Spring Harbor Laboratory, NY, 1982.

Kriegler and Botchan, Mol. Cell. Biol., 3:325, 1983.

Kriegler et al., Cell, 38:483, 1984.

Kriegler et al., Cell, 53:45, 1988.

Kuhl et al., Cell, 50:1057, 1987.

Kunz et al., Nucl. Acids Res., 17:1121, 1989.

Lareyre et al., J Biol Chem, 274(12):8282-90, 1999.

Larsen et al., Proc Natl. Acad. Sci. USA., 83:8283, 1986.

Laspia et al., Cell, 59:283, 1989.

Latimer et al., Mol. Cell. Biol., 10:760, 1990.

Laughlin et al., J Virol, 60(2):515-524, 1986.

Lebkowski et al., Mol. Cell. Biol., 8(10):3988-3996, 1988.

Lee et al., Nature, 294:228, 1981.

Lee et al., Nucleic Acids Res., 12:4191-206, 1984.

Lee et al., DNA Cell Biol, 16(11):1267-75, 1997.

Levenson et al., Hum Gene Ther. 20;9(8):1233-1236, 1998.

Levinson et al., Nature, 295:79, 1982.

Levy et al., Annu. Rev. Immunol., 16:89-109, 1998.

Lin et al., Mol. Cell. Biol., 10:850, 1990.

Luria et al., EMBO J., 6:3307, 1987.

Lusky and Botchan, Proc Natl. Acad. Sci. U.S.A., 83:3609, 1986.

Lusky et al., Mol. Cell. Biol. 3:1108, 1983.

Lusso, Virology, 273:228-240, 2000.

Macejak and Sarnow, Nature, 353:90-94, 1991.

Major and Feinstone, Hepatology, 25:1527-1538, 1997.

Majors and Varmus, Proc. Nat'l Acad. Sci. USA, 80:5866, 1983.

Mann et al., Cell, 33:153-159, 1983.

Marks et al., In: Basic Medical Biochemistry,. Williams and Wilkins, Baltimore, 1996.

McLaughlin et al., J Virol, 62(6):1963-1973, 1988.

McNeall et al., Gene, 76:81, 1989.

Merrifield, Science, 232: 341-347, 1986.

Miksicek et al., Cell, 46:203, 1986.

Miller et al., Am J Clin Oncol, 15(3):216-221, 1992.

Monazahian et al., J. Med. Virol. 57:223-229, 1999.

Monazahian et al., Med. Microbiol. Immunol., 188:177-184, 2000.

Mordacq and Linzer, Genes and Dev., 3:760, 1989.

Moreau et al., Nucl. Acids Res., 9:6047, 1981.

Muesing et al., Cell, 48:691, 1987.

Muzyczka, Curr Top Microbiol Immunol, 158:97-129, 1992.

Ng et al., Nuc. Acids Res., 17:601, 1989.

Nicolas and Rubinstein, In: Vectors: A survey of molecular cloning vectors and their uses, Rodriguez and Denhardt, eds., Stoneham: Butterworth, pp. 494-513, 1988.

Nicolau and Sene, Biochim. Biophys. Acta, 721:185-190, 1982.

Nicolau et al., Methods Enzymol., 149:157-176, 1987.

Nomoto et al., Gene, 236(2):259-71, 1999.

Ondek et al., EMBO J., 6:1017,1987.

Omitz et al., Mol. Cell. Biol., 7:3466, 1987.

Owsianka et al., J. Gen. Virol, 82:1877-1883, 2001.

Palmiter et al., Nature, 300:611, 1982.

Paskind et al., Virology, 67:242-248, 1975.

Patel et al., J. Gen. Virol., 81:2873-2883, 2000.

PCT Patent Publication WO 84/03564

PCT Patent Publication WO 94/09699

PCT Patent Publication WO/90/07582

PCT Patent Publication WO/91/00868

PCT Patent Publication WO/91/07087

PCT Patent Publication WO 90/11089

PCT Patent Publication WO 90/14436

Pech et al., Mol. Cell. Biol., 9:396, 1989.

Pelletier and Sonenberg, Nature, 334:320-325, 1988.

Perez-Stable and Constantini, Mol Cell. Biol., 10:1116, 1990.

Petracca et al., J. Virol., 74:4824-4830, 2000.

Picard and Schaffner, Nature, 307:83, 1984.

Pileri et al., Science, 282:938-941, 1998.

Pinkert et al., Genes and Dev., 1:268, 1987.

Plotkin et al., Liver Transplantation 6:S53-S56, 2000.

Polgreen et al., International AIDS Conference, Barcelona, Spain Abstract, 2002.

Ponta et al., Proc. Nat'l Acad. Sci. USA, 82:1020, 1985.

Porton et al., Mol. Cell. Biol., 10:1076, 1990.

Potter et al., Proc. Nat'l Acad. Sci. USA, 81:7161-7165, 1984.

Prince et al., J. Viral. Hepat., 3:11-17, 1996.

Queen and Baltimore, Cell, 35:741, 1983.

Quinn et al., Mol. Cell. Biol., 9:4713, 1989.

Redondo et al., Science, 247:1225, 1990.

Reisman and Rotter, Mol. Cell. Biol., 9:3571, 1989.

Remington's Pharmaceutical Sciences, 18th Ed. Mack Printing Company, 1990.

Resendez Jr. et al., Mol. Cell. Biol., 8:4579, 1988.

Ridgeway, In: Vectors: A survey of molecular cloning vectors and their uses. Rodriguez and Denhardt, eds. Stoneham: Butterworth, pp. 467-492, 1988.

Ripe et al., Mol. Cell. Biol., 9:2224, 1989.

Rippe et al., Mol. Cell Biol., 10:689-695, 1990.

Rittling et al., Nuc. Acids Res., 17:1619, 1989.

Rosen et al., Cell, 41:813, 1988.

Sakai et al., Genes and Dev., 2:1144, 1988.

Sambrook et al., In: Molecular cloning, Cold Spring Harbor Laboratory Press, Cold Spring Harbor, N.Y., 1989.

Satake et al., J. Virology, 62:970, 1988.

Schaffner et al., J. Mol. Biol., 201:81, 1988.

Scheider-Schaulies, J. Gen. Virol., 81:1413-1429, 2000.

Schmidt et al., J. Med. Virol., 47:153-160, 1995.

Schmidt et al., J. Infect. Dis., 176:20-26, 1997.

Searle et al., Mol. Cell. Biol., 5:1480, 1985.

Seeff, Hepatology, 26:21S-28S, 1997.

Sharp and Marciniak, Cell, 59:229, 1989.

Shaul and Ben-Levy, EMBO J., 6:1913, 1987.

Sherman et al., Mol. Cell. Biol., 9:50, 1989.

Shimizu et al., Proc. Natl. Acad. Sci. USA, 90:6037-6041, 1993.

Sleigh and Lockett, J. EMBO, 4:3831, 1985.

Spalholz et al., Cell, 42:183, 1985.

Spandau and Lee, J. Virology, 62:427, 1988.

Spandidos and Wilkie, EMBO J., 2:1193, 1983.

Stapleton et al., J. Clin. Microbiol., 37:484-489, 1999.

Stapleton et al., International AIDS Conference, Barcelona, Spain Abstract, 2002.

Stein et al., Circulation 104:257-262, 2001.

Stephens and Hentschel, Biochem. J, 248:1, 1987.

Stewart and Young, In: Solid Phase Peptide Synthesis, 2d. ed., Pierce Chem. Co., 1984.

Stuart et al., Nature, 317:828, 1985.

Sullivan and Peterlin, Mol. Cell. Biol., 7:3315, 1987.

Swartzendruber and Lehman, J. Cell. Physiology, 85:179, 1975.

Takebe et al., Mol Cell. Biol., 8:466, 1988.

Tam et al., J. Am. Chem. Soc., 105:6442, 1983.

Tavernier et al., Nature, 301:634, 1983.

Taylor and Kingston, Mol. Cell. Biol., 10:165, 1990a.

Taylor and Kingston, Mol. Cell. Biol., 10:176, 1990b.

Taylor et al., J. Biol. Chem., 264:15160, 1989.

Temin, In: Gene Transfer, Kucherlapati (ed.), New York: Plenum Press, pp. 149-188, 1986.

Thiesen et al., J. Virology, 62:614, 1988.

Thomas et al., J. Inf. Dis., 174:690-695, 1996.

Thomssen et al., Med. Microbiol. Immunol., 181:293-300, 1992.

Thomssen et al., Med. Microbiol. Immunol., 182:329-334, 1993.

Tratschin et al., Mol. Cell. Biol., 4:2072-2081, 1984.

Treisman, Cell, 42:889, 1985.

Tronche et al., Mol. Biol. Med., 7:173, 1990.

Trudel and Constantini, Genes and Dev. 6:954, 1987.

Tsumaki et al., J Biol Chem, 273(36):22861-4, 1998.

Tur-Kaspa et al., Mol. Cell Biol., 6:716-718, 1986.

Tyndell et al., Nuc. Acids. Res., 9:6231, 1981.

Vannice and Levinson, J. Virology, 62:1305, 1988.

Vasseur et al., Proc Natl. Acad. Sci. U.S.A., 77:1068, 1980.

Wang and Calame, Cell, 47:241, 1986.

Wang et al., J. Clin. Invest., 91:1380-1389, 1993.

Weber et al., Cell, 36:983, 1984.

Weinberger et al. Mol. Cell. Biol., 8:988, 1984.

Williams, Am. J. Med., 107:2S-9S, 1999.

Wilson et al., Science, 244:1344-1346, 1989.

Winoto and Baltimore, Cell 59:649, 1989.

Wolfe et al., 9th Conference on Retroviruses and Opportunistic Infections, Seattle, 2002.

Wong et al., Gene, 10:87-94, 1980.

Wu and Wu, J. Biol. Chem., 262:4429-4432, 1987.

Wu and Wu, Biochemistry, 27:887-892, 1988.

Wu et al., Biochem Biophys Res Commun, 233(1):221-6, 1997.

Wuenschmann et al., J. Virol., 74:10055-10062, 2000.

Xiang et al., J. Viral. Hepat., 6:S16-S22, 1999.

Xiang et al., J. Virol., 72:2738-2744, 1998.

Xiang et al., N. Engl. J. Med., 345:707-714, 2001.

Yutzey et al. Mol. Cell. Biol., 9:1397, 1989.

Zhao-Emonet et al., Biochim Biophys Acta, 1442(2-3):109-19, 1998.

Zufferey et al., Nat Biotechnol, 15(9):871-875, 1997.

What is claimed is:

1. A method of identifying an inhibitor of Hepatitis C Virus (HCV) infection comprising:
(a) providing isolated E2 glycoprotein and plasma lipoprotein;
(b) admixing a candidate substance with the E2 glycoprotein and plasma lipoprotein; and
(c) determining the binding of the E2 glycoprotein to plasma lipoprotein,
wherein a reduction in E2 glycoprotein binding to plasma lipoprotein, as compared to binding in the absence of the candidate substance, identifies the candidate substance as an inhibitor of HCV infection.

2. The method of claim 1, wherein the candidate substance is an anti-E2 antibody.

3. The method of claim 2, wherein the anti-E2 antibody is a monoclonal antibody.

4. The method of claim 2, wherein the anti-E2 antibody is a polyclonal antibody.

5. The method of claim 1, wherein the inhibitor is a small molecule.

6. The method of claim 1, wherein the inhibitor is a peptide.

7. The method of claim 1, wherein the binding of the E2 glycoprotein to plasma lipoprotein is determined by gel electrophoresis, gel filtration chromatography, fluorescence quenching assay, flow cytometry, elisa, solid phase immunoassay, or confocal microscopy.

8. A method of identifying an inhibitor of Hepatitis C Virus (HCV) infection comprising:
(a) providing isolated E2 glycoprotein and plasma lipoprotein under conditions effective to allow the formation of an E2 glycoprotein/plasma lipoprotein complex;
(b) providing a target cell expressing an LDL receptor;
(c) admixing said E2 glycoprotein/plasma lipoprotein complex and said target cell in the presence of a candidate substance; and
(d) determining the binding of the E2 glycoprotein/plasma lipoprotein complex to LDL receptor,
wherein a reduction in E2 glycoprotein/plasma lipoprotein complex binding to LDL receptor, as compared to binding in the absence of the candidate substance, identifies the candidate substance as an inhibitor of HCV infection.

9. The method of claim 8, wherein the candidate substance is an anti-LDL receptor antibody.

10. The method of claim 9, wherein the anti-LDL receptor antibody is a monoclonal antibody.

11. The method of claim 9, wherein the anti-LDL receptor antibody is a polyclonal antibody.

12. The method of claim 8, wherein the candidate substance is an anti-E2 glycoprotein/plasma lipoprotein antibody.

13. The method of claim 12, wherein the anti-LDL receptor antibody is a monoclonal antibody.

14. The method of claim 12, wherein the anti-LDL receptor antibody is a polyclonal antibody.

15. The method of claim 8, wherein the inhibitor is a small molecule.

16. The method of claim 8, wherein the inhibitor is a peptide.

17. The method of claim 8, wherein the binding of the E2 glycoprotein/plasma lipoprotein complex to LDL receptor is determined by gel electrophoresis, gel filtration chromatography, fluorescence quenching assay, flow cytometry, elisa, solid phase immunoassay, or confocal microscopy.

18. A method of identifying an inhibitor of Hepatitis C Virus (HCV) infection comprising:
(a) providing isolated E2 glycoprotein, plasma lipoprotein, and a target cell expressing an LDL receptor under conditions effective to allow the formation of an E2 glycoprotein/plasma lipoprotein/LDL receptor complex;
(b) contacting the LDL-expressing cell with a candidate substance; and
(c) determining internalization of E2 glycoprotein/plasma lipoprotein/LDL receptor complex into target cell,
wherein a reduction in internalization of E2 glycoprotein/plasma lipoprotein/LDL receptor complex into target cell, as compared to internalization of E2 glycoprotein/plasma lipoprotein/LDL receptor complex into target cell in the absence of the candidate substance, identifies the candidate substance as an inhibitor of HCV infection.

19. The method of claim 18, wherein the candidate substance is an antibody.

20. The method of claim 19, wherein the antibody is a monoclonal antibody.

21. The method of claim 19, wherein the antibody is a polyclonal antibody.

22. The method of claim 18, wherein the inhibitor is a small molecule.

23. The method of claim 18, wherein the inhibitor is a peptide.

24. A method of removing plasma lipoproteins from a blood sample comprising:
(a) providing isolated E2 glycoprotein attached to a support;
(b) contacting the support with the blood sample under conditions effective to allow the binding of plasma lipoprotein present in the blood sample to E2 glycoprotein; and
(c) separating plasma lipoprotein from E2 glycoprotein.

25. The method of claim 24, wherein the support is a non-reactive solid support.

26. The method of claim 25, wherein the non-reactive solid support is nitrocellulose membrane, a bead support, or a glass support.

27. A method of screening for an inhibitor of Hepatitis C Virus (HCV) infection comprising:
(a) providing purified E2 glycoprotein and plasma lipoprotein;
(b) admixing an E2 antibody with E2 glycoprotein and plasma lipoprotein under conditions effective to allow the formation of an E2 glycoprotein/plasma lipoprotein complex; and
(c) determining the binding of E2 glycoprotein to plasma lipoprotein,
wherein a reduction in E2 glycoprotein binding to plasma lipoprotein, as compared to binding in the absence of E2 antibody, identifies the E2 antibody as an inhibitor of HCV infection.

28. The method of claim 27, wherein the binding of the E2 glycoprotein to plasma lipoprotein is determined by gel electrophoresis, gel filtration chromatography, fluorescence quenching assay, flow cytometry, elisa, solid phase immunoassay, or confocal microscopy.

29. A method of inhibiting Hepatitis C Virus (HCV) infection in a subject comprising administering an effective amount of an agent that inhibits the formation of an E2 glycoprotein/plasma lipoprotein complex or an E2 glycoprotein/plasma lipoprotein/LDL receptor complex.

30. The method of claim 29, wherein the agent is a small molecule.

31. The method of claim 29, wherein the agent is a peptide.

32. The method of claim 29, wherein the agent is an antibody.

33. The method of claim 32, wherein the antibody is a polyclonal antibody.

34. The method of claim 50, wherein the antibody is a monoclonal antibody.

35. The method of claim 29, wherein the administration of the agent is by oral administration.

36. The method of claim 29, wherein the administration of the agent is by intravenous administration.

37. The method of claim 29, wherein the administration of the agent is by parenteral administration.

38. The method of claim 29, wherein the administration of the agent is by intramuscular administration.

39. The method of claim 29, wherein the agent is formulated in an aqueous formulation.

40. The method of claim 29, wherein the agent is formulated in a salt formulation.

41. The method of claim 29, wherein the agent is formulated as an ingestible tablet, capsule, elixir, suspension, syrup, or a wafer.

42. The method of claim 29, further comprising administering in combination with said agent that inhibits the formation of an E2 glycoprotein/plasma lipoprotein complex, another agent effective in treating HCV infection in a subject.

43. The method of claim 29, further comprising administering in combination with said agent that inhibits the formation of an E2 glycoprotein/plasma lipoprotein/LDL receptor complex, another agent effective in treating HCV infection in a subject.

44. A method of inhibiting Hepatitis C Virus (HCV) infection in a subject comprising administering an effective amount of an agent that inhibits the internalization of E2 glycoprotein/plasma lipoprotein/LDL receptor complex into a target cell.

45. The method of claim 44, wherein the agent is a small molecule.

46. The method of claim 44, wherein the agent is a peptide.

47. The method of claim 44, wherein the agent is an antibody.

48. The method of claim 47, wherein the antibody is a polyclonal antibody.

49. The method of claim 47, wherein the antibody is a monoclonal antibody.

50. The method of claim 44, wherein the administration of the agent is by oral administration.

51. The method of claim 44, wherein the administration of the agent is by intravenous administration.

52. The method of claim 44, wherein the administration of the agent is by parenteral administration.

53. The method of claim 44, wherein the administration of the agent is by intramuscular administration.

54. The method of claim 44, wherein the agent is formulated in an aqueous formulation.

55. The method of claim 44, wherein the agent is formulated in a salt formulation.

56. The method of claim 44, wherein the agent is formulated as an ingestible tablet, capsule, elixir, suspension, syrup, or a wafer.

57. The method of claim 44, further comprising administering in combination with said agent that inhibits the internalization of E2 glycoprotein/plasma lipoprotein/LDL receptor complex into a target cell, another agent effective in treating HCV infection in a subject.

58. An inhibitor of Hepatitis C Virus (HCV) infection that reduces or prevents the formation of an E2 glycoprotein/plasma lipoprotein complex, or an E2 glycoprotein/plasma lipoprotein/LDL receptor complex.

59. The inhibitor of claim 58, wherein the inhibitor is a small molecule.

60. The inhibitor of claim 58, wherein the inhibitor is a peptide.

61. The inhibitor of claim 58, wherein the inhibitor is an antibody.

62. The antibody of claim 61, wherein the antibody is a polyclonal antibody.

63. The antibody of claim 61, wherein the antibody is a monoclonal antibody.

64. An inhibitor of Hepatitis C Virus (HCV) infection that reduces or prevents the internalization of an E2 glycoprotein/plasma lipoprotein/LDL receptor complex into a target cell.

65. The inhibitor of claim 64, wherein the inhibitor is a small molecule.

66. The inhibitor of claim 64, wherein the inhibitor is a peptide.

67. The inhibitor of claim 64, wherein the inhibitor is an antibody.

68. The inhibitor of claim 67, wherein the antibody is a polyclonal antibody or a monoclonal antibody.

69. A method for reducing LDL levels in a subject comprising administering to said subject an HCV E2 glycoprotein.

70. The method of claim 69, wherein said E2 glycoprotein is substantially purified away from other HCV components.

71. The method of claim 69, wherein said E2 glycoprotein is comprised in a non-replicative viral particle.

72. The method of claim 69, wherein said subject has a history of familial hypercholesterolemia.

73. The method of claim 69, wherein said HCV E2 glycoprotein is administered intravenously.

74. The method of claim 69, further comprising administering in combination with said E2 glycoprotein, another agent effective in lowering LDL levels in a subject.

75. The method of claim 74, wherein said agent is selected from the group consisting of nicotinic acid, clofibrate, dextrothyroxine sodium, neomycin, beta-sitosterol, probucol, cerivastatin, fluvastatin, atorvastatin, lovastatin, pravastatin, simvastatin, cholestyramine and HMG-CoA reductase inhibitors.

76. A method of identifying an E2 peptide that is effective in lowering LDL levels in a subject comprising:
(a) providing a candidate E2 peptide, plasma lipoprotein, and a target cell expressing an LDL receptor under conditions effective to allow the formation of an E2 peptide/plasma lipoprotein/LDL receptor complex,
(b) assaying internalization of E2 peptide/plasma lipoprotein/LDL receptor complex,
wherein an increase in plasma lipoprotein into target cell, as compared to internalization of plasma lipoprotein into target cell in the absence of the E2 peptide, identifies the E2 peptide as effective in lowering LDL levels in a subject.

77. The method of claim 76, wherein the E2 peptide is a produced by chemical, physical or enzymatic cleavage of a purified E2 glycoprotein.

78. The method of claim 76, wherein the E2 peptide is a C-terminal truncated E2 molecule.

79. The method of claim 76, wherein the E2 peptide is a recombinant peptide.

80. The method of claim 76, wherein the E2 peptide is chemically synthesized.

81. The method of claim 76, wherein the internalization of the E2 peptide/plasma lipoprotein/LDL receptor complex into target cell is determined by labeling the plasma lipoprotein with a label.

82. The method of claim 76, wherein the label is selected from the group consisting of radio label, isotopic label, fluorescent label, enzymatic label and chemiluminescent label.

83. The method of claim 76, wherein the plasma lipoprotein is low density lipoprotein.

84. The method of claim 76, wherein the subject is a human.


SEQUENCE LISTING (3 sequences)

SEQ ID NO: 1 - DNA, 9401 bp, Hepatitis C virus
  CDS (342)..(9374)
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaaaaaaaaacaaacgtaacaccaaccgtcgcccacaggacgtcaagttcccgggtggcggtcagatcgttggtggagtttacttgttgccgcgcaggggccctagattgggtgtgcgcgcgacgagaaagacttccgagcggtcgcaacctcgaggtagacgtcagcctatccccaaggctcgtcggcccgagggcaggacctgggctcagcccgggtacccttggcccctctatggcaatgagggctgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcttgactgtgcccgcttcggcctaccaagtgcgcaactccacggggctttaccacgtcaccaatgattgccctaactcgagtattgtgtacgaggcggccgatgccatcctgcacactccggggtgcgtcccttgcgttcgtgagggcaacgcctcgaggtgttgggtggcgatgacccctacggtggccaccagggatggcaaactccccgcgacgcagcttcgacgtcacatcgatctgcttgtcgggagcgccaccctctgttcggccctctacgtgggggacctatgcgggtctgtctttcttgtcggccaactgttcaccttctctcccaggcgccactggacgacgcaaggttgcaattgctctatctatcccggccatataacgggtcaccgcatggcatgggatatgatgatgaactggtcccctacgacggcgttggtaatggctcagctgctccggatcccacaagccatcttggacatgatcgctggtgctcactggggagtcctggcgggcatagcgtatttctccatggtggggaactgggcgaaggtcctggtagtgctgctgctatttgccggcgtcgacgcggaaacccacgtcaccgggggaagtgccggccacactgtgtctggatttgttagcctcctcgcaccaggcgccaagcagaacgtccagctgatcaacaccaacggcagttggcacctcaatagcacggccctgaactgcaatgatagcctcaacaccggctggttggcagggcttttctatcaccacaagttcaactcttcaggctgtcctgagaggctagccagctgccgaccccttaccgattttgaccagggctggggccctatcagttatgccaacggaagcggccccgaccagcgcccctactgctggcactaccccccaaaaccttgcggtattgtgcccgcgaagagtgtgtgtggtccggtatattgcttcactcccagccccgtggtggtgggaacgaccgacaggtcgggcgcgcccacctacagctggggtgaaaatgatacggacgtcttcgtccttaacaataccaggccaccgctgggcaattggttcggttgtacctggatgaactcaactggattcaccaaagtgtgcggagcgcctccttgtgtcatcggaggggcgggcaacaacaccctgcactgccccactgattgcttccgcaagcatccggacgccacatactctcggtgcggctccggtccctggatcacacccaggtgcctggtcgactacccgtataggctttggcattatccttgtaccatcaactacaccatatttaaaatcaggatgtacgtgggaggggtcgaacacaggctggaagctgcctgcaactggacgcggggcgaacgttgcgatctggaagacagggacaggtccgagctcagcccgttactgctgaccactacacagtggcaggtcctcccgtgttccttcacaaccctaccagccttgtccaccggcctcatccacctccaccagaacattgtggacgtgcagtacttgtacggggtggggtcaagcatcgcgtcctgggccattaagtgggagtacgtcgttctcctgttccttctgcttgcagacgcgcgcgtctgctcctgcttgtggatgatgctactcatatcccaagcggaggcggctttggagaacctcgtaatacttaatgcagcatccctggccgggacgcacggtcttgtatccttcctcgtgttcttctgctttgcatggtatttgaagggtaagtgggtgcccggagcggtctacaccttctacgggatgtggcctctcctcctgctcctgttggcgttgccccagcgggcgtacgcgctggacacggaggtggccgcgtcgtgtggcggtgttgttctcgtcgggttgatggcgctgactctgtcaccatattacaagcgctatatcagctggtgcttgtggtggcttcagtattttctgaccagagtggaagcgcaactgcacgtgtggattccccccctcaacgtccgaggggggcgcgacgccgtcatcttactcatgtgtgctgtacacccgactctggtatttgacatcaccaaattgctgctggccgtcttcggacccctttggattcttcaagccagtttgcttaaagtaccctactttgtgcgcgtccaaggccttctccggttctgcgcgttagcgcggaagatgatcggaggccattacgtgcaaatggtcatcattaagttaggggcgcttactggcacctatgtttataaccatctcactcctcttcgggactgggcgcacaacggcttgcgagatctggccgtggctgtagagccagtcgtcttctcccaaatggagaccaagctcatcacgtggggggcagataccgccgcgtgcggtgacatcatcaacggcttgcctgtttccgcccgcaggggccgggagatactgctcgggccagccgatggaatggtctccaaggggtggaggttgctggcgcccatcacggcgtacgcccagcagacaaggggcctcctagggtgcataatcaccagcctaactggccgggacaaaaaccaagtggagggtgaggtccagattgtgtcaactgctgcccaaaccttcctggcaacgtgcatcaatggggtgtgctggactgtctaccacggggccggaacgaggaccatcgcgtcacccaagggtcctgtcatccagatgtataccaatgtagaccaagaccttgtgggctggcccgctccgcaaggtagccgctcattgacaccctgcacttgcggctcctcggacctttacctggtcacgaggcacgccgatgtcattcccgtgcgccggcggggtgatagcaggggcagcctgctgtcgccccggcccatttcctacttgaaaggctcctcggggggtccgctgttgtgccccgcggggcacgccgtgggcatatttagggccgcggtgtgcacccgtggagtggctaaggcggtggactttatccctgtggagaacctagagacaaccatgaggtccccggtgttcacggataactcctctccaccagtagtgccccagagcttccaggtggctcacctccatgctcccacaggcagcggcaaaagcaccaaggtcccggctgcatatgcagctcagggctataaggtgctagtactcaacccctctgttgctgcaacactgggctttggtgcttacatgtccaaggctcatgggatcgatcctaacatcaggaccggggtgagaacaattaccactggcagccccatcacgtactccacctacggcaagttccttgccgacggcgggtgctcggggggcgcttatgacataataatttgtgacgagtgccactccacggatgccacatccatcttgggcatcggcactgtccttgaccaagcagagactgcgggggcgagactggttgtgctcgccaccgccacccctccgggctccgtcactgtgccccatcccaacatcgaggaggttgctctgtccaccaccggagagatccctttttacggcaaggctatccccctcgaagtaatcaagggggggagacatctcatcttctgtcattcaaagaagaagtgcgacgaactcgccgcaaagctggtcgcattgggcatcaatgccgtggcctactaccgcggtcttgacgtgtccgtcatcccgaccagcggcgatgttgtcgtcgtggcaaccgatgccctcatgaccggctataccggcgacttcgactcggtgatagactgcaatacgtgtgtcacccagacagtcgatttcagccttgaccctaccttcaccattgagacaatcacgctcccccaggatgctgtctcccgcactcaacgtcggggcaggactggcagggggaagccaggcatctacagatttgtggcaccgggggagcgcccctccggcatgttcgactcgtccgtcctctgtgagtgctatgacgcaggctgtgcttggtatgagctcacgcccgccgagactacagttaggctacgagcgtacatgaacaccccggggcttcccgtgtgccaggaccatcttgaattttgggagggcgtctttacaggcctcactcatatagatgcccactttctatcccagacaaagcagagtggggagaaccttccttacctggtagcgtaccaagccaccgtgtgcgctagggctcaagcccctcccccatcgtgggaccagatgtggaagtgtttgattcgcctcaagcccaccctccatgggccaacacccctgctatacagactgggcgctgttcagaatgaaatcaccctgacgcacccagtcaccaaatacatcatgacatgcatgtcggccgacctggaggtcgtcacgagcacctgggtgctcgttggcggcgtcctggctgctttggccgcgtattgcctgtcaacaggctgcgtggtcatagtgggcagggtcgtcttgtccgggaagccggcaatcatacctgacagggaagtcctctaccgagagttcgatgagatggaagagtgctctcagcacttaccgtacatcgagcaagggatgatgctcgccgagcagttcaagcagaaggccctcggcctcctgcagaccgcgtcccgtcaggcagaggttatcgcccctgctgtccagaccaactggcaaaaactcgagaccttctgggcgaagcatatgtggaacttcatcagtgggatacaatacttggcgggcttgtcaacgctgcctggtaaccccgccattgcttcattgatggcttttacagctgctgtcaccagcccactaaccactagccaaaccctcctcttcaacatattgggggggtgggtggctgcccagctcgccgcccccggtgccgctactgcctttgtgggcgctggcttagctggcgccgccatcggcagtgttggactggggaaggtcctcatagacatccttgcagggtatggcgcgggcgtggcgggagctcttgtggcattcaagatcatgagcggtgaggtcccctccacggaggacctggtcaatctactgcccgccatcctctcgcccggagccctcgtagtcggcgtggtctgtgcagcaatactgcgccggcacgttggcccgggcgagggggcagtgcagtggatgaaccggctgatagccttcgcctcccgggggaaccatgtttcccccacgcactacgtgccggagagcgatgcagctgcccgcgtcactgccatactcagcagcctcactgtaacccagctcctgaggcgactgcaccagtggataagctcggagtgtaccactccatgctccggttcctggctaagggacatctgggactggatatgcgaggtgttgagcgactttaagacctggctaaaagctaagctcatgccacagctgcctgggatcccctttgtgtcctgccagcgcgggtataagggggtctggcgagtggacggcatcatgcacactcgctgccactgtggagctgagatcactggacatgtcaaaaacgggacgatgaggatcgtcggtcctaggacctgcaggaacatgtggagtgggaccttccccattaatgcctacaccacgggcccctgtaccccccttcctgcgccgaactacacgttcgcgctatggagggtgtctgcagaggaatatgtggagataaggcaggtgggggacttccactacgtgacgggtatgactactgacaatctcaaatgcccgtgccaggtcccatcgcccgaatttttcacagaattggacggggtgcgcctacataggtttgcgcccccctgcaagcccttgctgcgggaggaggtatcattcagagtaggactccacgaatacccggtagggtcgcaattaccttgcgagcccgaaccggacgtggccgtgttgacgtccatgctcactgatccctcccatataacagcagaggcggccgggcgaaggttggcgaggggatcacccccctctgtggccagctcctcggctagccagctatccgctccatctctcaaggcaacttgcaccgctaaccatgactcccctgatgctgagctcatagaggccaacctcctatggaggcaggagatgggcggcaacatcaccagggttgagtcagaaaacaaagtggtgattctggactccttcgatccgcttgtggcggaggaggacgagcgggagatctccgtacccgcagaaatcctgcggaagtctcggagattcgcccaggccctgcccgtttgggcgcggccggactataaccccccgctagtggagacgtggaaaaagcccgactacgaaccacctgtggtccatggctgtccgcttccacctccaaagtcccctcctgtgcctccgcctcggaagaagcggacggtggtcctcactgaatcaaccctatctactgccttggccgagctcgccaccagaagctttggcagctcctcaacttccggcattacgggcgacaatacgacaacatcctctgagcccgccccttctggctgcccccccgactccgacgctgagtcctattcctccatgccccccctggagggggagcctggggatccggatcttagcgacgggtcatggtcaacggtcagtagtgaggccaacgcggaggatgtcgtgtgctgctcaatgtcttactcttggacaggcgcactcgtcaccccgtgcgccgcggaagaacagaaactgcccatcaatgcactaagcaactcgttgctacgtcaccacaatttggtgtattccaccacctcacgcagtgcttgccaaaggcagaagaaagtcacatttgacagactgcaagttctggacagccattaccaggacgtactcaaggaggttaaagcagcggcgtcaaaagtgaaggctaacttgctatccgtagaggaagcttgcagcctgacgcccccacactcagccaaatccaagtttggttatggggcaaaagacgtccgttgccatgccagaaaggccgtaacccacatcaactccgtgtggaaagaccttctggaagacaatgtaacaccaatagacactaccatcatggctaagaacgaggttttctgcgttcagcctgagaaggggggtcgtaagccagctcgtctcatcgtgttccccgatctgggcgtgcgcgtgtgcgaaaagatggctttgtacgacgtggttacaaagctccccttggccgtgatgggaagctcctacggattccaatactcaccaggacagcgggttgaattcctcgtgcaagcgtggaagtccaagaaaaccccaatggggttctcgtatgatacccgctgctttgactccacagtcactgagagcgacatccgtacggaggaggcaatctaccaatgttgtgacctcgacccccaagcccgcgtggccatcaagtccctcaccgagaggctttatgttgggggccctcttaccaattcaaggggggagaactgcggctatcgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagcgcgggggtccaggaggacgcggcgagcctgagagccttcacggaggctatgaccaggtactccgccccccctggggaccccccacaaccagaatacgacttggagctcataacatcatgctcctccaacgtgtcagtcgcccacgacggcgctggaaagagggtctactacctcacccgtgaccctacaacccccctcgcgagagctgcgtgggagacagcaagacacactccagtcaattcctggctaggcaacataatcatgtttgcccccacactgtgggcgaggatgatactgatgacccatttctttagcgtccttatagccagggaccagcttgaacaggccctcgattgcgagatctacggggcctgctactccatagaaccacttgatctacctccaatcattcaaagactccatggcctcagcgcattttcactccacagttactctccaggtgaaattaatagggtggccgcatgcctcagaaaacttggggtaccgcccttgcgagcttggagacaccgggcccggagcgtccgcgctaggcttctggccagaggaggcagggctgccatatgtggcaagtacctcttcaactgggcagtaagaacaaagctcaaactcactccaatagcggccgctggccagctggacttgtccggctggttcacggctggctacagcgggggagacatttatcacagcgtgtctcatgcccggccccgctggatctggttttgcctactcctgcttgctgcaggggtaggcatctacctcctccccaaccgatgaaggttggggtaaacactccggcct

SEQ ID NO: 2 - protein, 3011 aa, Hepatitis C virus
MSTNPKPQKKNKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSTGLYHVTNDCPNSSIVYEAADAILHTPGCVPCVREGNASRCWVAMTPTVATRDGKLPATQLRRHIDLLVGSATLCSALYVGDLCGSVFLVGQLFTFSPRRHWTTQGCNCSIYPGHITGHRMAWDMMMNWSPTTALVMAQLLRIPQAILDMIAGAHWGVLAGIAYFSMVGNWAKVLVVLLLFAGVDAETHVTGGSAGHTVSGFVSLLAPGAKQNVQLINTNGSWHLNSTALNCNDSLNTGWLAGLFYHHKFNSSGCPERLASCRPLTDFDQGWGPISYANGSGPDQRPYCWHYPPKPCGIVPAKSVCGPVYCFTPSPVVVGTTDRSGAPTYSWGENDTDVFVLNNTRPPLGNWFGCTWMNSTGFTKVCGAPPCVIGGAGNNTLHCPTDCFRKHPDATYSRCGSGPWITPRCLVDYPYRLWHYPCTINYTIFKIRMYVGGVEHRLEAACNWTRGERCDLEDRDRSELSPLLLTTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGKWVPGAVYTFYGMWPLLLLLLALPQRAYALDTEVAASCGGVVLVGLMALTLSPYYKRYISWCLWWLQYFLTRVEAQLHVWIPPLNVRGGRDAVILLMCAVHPTLVFDITKLLLAVFGPLWILQASLLKVPYFVRVQGLLRFCALARKMIGGHYVQMVIIKLGALTGTYVYNHLTPLRDWAHNGLRDLAVAVEPVVFSQMETKLITWGADTAACGDIINGLPVSARRGREILLGPADGMVSKGWRLLAPITAYAQQTRGLLGCIITSLTGRDKNQVEGEVQIVSTAAQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGIFRAAVCTRGVAKAVDFIPVENLETTMRSPVFTDNSSPPVVPQSFQVAHLHAPTGSGKSTKVPAAYAAQGYKVLVLNPSVAATLGFGAYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGYTGDFDSVIDCNTCVTQTVDFSLDPTFTIETITLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEITLTHPVTKYIMTCMSADLEVVTSTWVLVGGVLAALAAYCLSTGCVVIVGRVVLSGKPAIIPDREVLYREFDEMEECSQHLPYIEQGMMLAEQFKQKALGLLQTASRQAEVIAPAVQTNWQKLETFWAKHMWNFISGIQYLAGLSTLPGNPAIASLMAFTAAVTSPLTTSQTLLFNILGGWVAAQLAAPGAATAFVGAGLAGAAIGSVGLGKVLIDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYKGVWRVDGIMHTRCHCGAEITGHVKNGTMRIVGPRTCRNMWSGTFPINAYTTGPCTPLPAPNYTFALWRVSAEEYVEIRQVGDFHYVTGMTTDNLKCPCQVPSPEFFTELDGVRLHRFAPPCKPLLREEVSFRVGLHEYPVGSQLPCEPEPDVAVLTSMLTDPSHITAEAAGRRLARGSPPSVASSSASQLSAPSLKATCTANHDSPDAELIEANLLWRQEMGGNITRVESENKVVILDSFDPLVAEEDEREISVPAEILRKSRRFAQALPVWARPDYNPPLVETWKKPDYEPPVVHGCPLPPPKSPPVPPPRKKRTVVLTESTLSTALAELATRSFGSSSTSGITGDNTTTSSEPAPSGCPPDSDAESYSSMPPLEGEPGDPDLSDGSWSTVSSEANAEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVTHINSVWKDLLEDNVTPIDTTIMAKNEVFCVQPEKGGRKPARLIVFPDLGVRVCEKMALYDVVTKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALDCEIYGACYSIEPLDLPPIIQRLHGLSAFSLHSYSPGEINRVAACLRKLGVPPLRAWRHRARSVRARLLARGGRAAICGKYLFNWAVRTKLKLTPIAAAGQLDLSGWFTAGYSGGDIYHSVSHARPRWIWFCLLLLAAGVGIYLLPNR

SEQ ID NO: 3 - protein, 333 aa, Hepatitis C virus
AETHVTGGSAGHTVSGFVSLLAPGAKQNVQLINTNGSWHLNSTALNCNDSLNTGWLAGLFYHHKFNSSGCPERLASCRPLTDFDQGWGPISYANGSGPDQRPYCWHYPPKPCGIVPAKSVCGPVYCFTPSPVVVGTTDRSGAPTYSWGENDTDVFVLNNTRPPLGNWFGCTWMNSTGFTKVCGAPPCVIGGAGNNTLHCPTDCFRKHPDATYSRCGSGPWITPRCLVDYPYRLWHYPCTINYTIFKIRMYVGGVEHRLEAACNWTRGERCDLEDRDRSELSPLLLTTTQWQVLPCSFTTLPALSTGLIHLHQNIVDVQYLYGVGSSIASWAIK